(19) United States
(12) Patent Application Publication
(10) Pub. No.: US 2015/0119851 A1
(43) Pub. Date: Apr. 30, 2015

(54) INSTRUMENT AND METHOD FOR DELIVERY, DEPLOYMENT, AND TAMPONADE OF HEMOSTATS AND METHODS OF ASSEMBLING AN INSTRUMENT THEREFOR

(71) Applicant: Nordson Corporation, Westlake, OH (US)

(72) Inventors: Jon E. Hoogenakker, Inver Grove Heights, MN (US); Thomas A. Kirk, Hastings, MN (US); Huadong Lou, Plymouth, MN (US); Bradley D. Robb, Maple Plain, MN (US)

(21) Appl. No.: 14/517,161

(22) Filed: Oct. 17, 2014

Related U.S. Application Data
(60) Provisional application No. 61/897,512, filed on Oct. 30, 2013.

(51) Int. Cl. A61B 17/12

(52) U.S. Cl. CPC A61B 17/12131; A61B 2017/12004; A61B 2017/1205
USPC 604/507; 604/264

(57) ABSTRACT

An instrument for delivery, deployment, and tamponade of a hemostat. A delivery cannula is included and has a proximal end, a distal end, and a lumen therebetween. A shaft is configured to be introduced into and move relative to the lumen. A hemostat applicator at the distal end of the shaft includes a tamponade surface. The applicator is configured to transition between a first, compact state for introduction into and movement within the lumen, and a second, expanded state as the applicator exits from the delivery cannula.

### CROSS-REFERENCE TO RELATED APPLICATION

This application claims the priority of U.S. Provisional Patent Application Ser. No. 61/897,512, filed Oct. 30, 2013 (pending), the disclosure of which is incorporated by reference herein.

### TECHNICAL FIELD

The disclosure relates generally to controlling bleeding, and is more specifically related to delivery, deployment, and tamponade of hemostats.

### BACKGROUND OF THE INVENTION

Medical textiles are used during surgical procedures to control bleeding, minimize blood loss, reduce post-surgical complications, and shorten the duration of surgery. Commonly used medical textiles include adhesion barriers, sponges, meshes, and hemostatic wound dressings that are applied to the surface of tissue. Medical textiles have typically been delivered to surgical sites using grasping instruments such as clamps and forceps. There have been attempts to use applicator instruments for delivering medical textiles.

For example, U.S. Patent Publication No. 2009/0234380 (now issued U.S. Pat. No. 8,372,092) discloses an applicator instrument including an outer shaft and an intermediate shaft received within the outer shaft. The distal end of the outer shaft is covered by a seal element, which receives a folded medical textile through use of a separate element in the form of a cartridge loader. The seal element also receives the distal end of the intermediate shaft such that the distal end of the intermediate shaft is covered by the seal element but is movable relative thereto. Once the seal element is placed against target tissue, an actuator mechanism, such as a trigger, is pulled by a user to cause the distal movement of the shaft to pierce the seal member and deliver the still-folded textile against the target tissue. Then, a second actuator, such as a plunger of a syringe, is actuated to inflate a balloon which acts as a tamponade surface. In order that the tamponade surface of the balloon includes a large enough surface area to tamponade the entire textile, a third actuator must be actuated in order to change the shape of the balloon and thus the tamponade surface. In order to retract the intermediate shaft, the second actuator is actuated to deflate the balloon and the instrument may be retracted from the trocar.

U.S. Pat. No. 5,397,332 discloses an applicator for applying a sheet of surgical material, such as surgical mesh, to internal body tissue. The applicator includes a delivery tube, a deployment tube slidably received within the delivery tube, and a shaft or irrigation tube slidably received within the deployment tube. An expandable spreader tip is connected between the distal end of the shaft and the distal end of the deployment tube. The spreader tip is collapsed and inserted in the delivery tube with the surgical mesh. The applicator is inserted through a trocar into a body cavity and the spreader tip is exposed by retracting the delivery tube relative to the deployment tube and shaft. The applicator has a first shaft for urging the spreader tip and surgical mesh into engagement with the tissue as the deployment tube is retracted, and a second actuator for advancing the deployment tube relative to the shaft to expand the spreader tip to apply the surgical mesh to the tissue. The spreader tip is biased towards the contracted position, even when it is exposed and outside of the deployment tube, such that it expands only upon actuation of the second actuator. In that regard, the applicator includes a return spring to bias the deployment tube proximally relative to the shaft to normally maintain the spreader tip in a collapsed configuration regardless of its position (inside or outside of) relative to the delivery tube.

Each of these applicators and others in the prior art rely on somewhat complicated mechanisms with several actuators in one or more of the delivery, deployment, and tamponade stages. Some even require ancillary parts that increase the complexity, as well as the costs to manufacture the devices. Moreover, because the hemostat is automatically deployed upon activating an advancement mechanism, there is a risk of misplacement. Therefore, an additional access port into the abdomen would be needed regardless, due to the risk of misplacement, to allow the insertion of a grabber or other tool in order to place the hemostat correctly. There is, therefore, a need in the art for improving and simplifying instruments and methods for the delivery, deployment, and tamponade of hemostats.

### SUMMARY

To that end, an instrument that relies on simple relative movement between an inner delivery shaft and an outer delivery cannula for the delivery, deployment, and tamponade of a hemostat is provided. In one embodiment, an instrument for delivery, deployment, and tamponade of a hemostat is provided and includes a delivery cannula having a proximal end, a distal end, and a lumen therebetween; a shaft having a distal end and being configured to be introduced into and move relative to the lumen; and a hemostat applicator at the distal end of the shaft and including a tamponade surface, the applicator being configured to transition between a first, compact state for introduction into and movement within the lumen, and a second, expanded state as the applicator exits from the delivery cannula.

In another embodiment, an instrument for delivery, deployment, and tamponade of a hemostat is provided and includes a delivery cannula having a proximal end, a distal end, and a lumen therebetween; a shaft having a distal end and being configured to be introduced into and move relative to the lumen; and a hemostat applicator coupled to the distal end of the shaft and including a tamponade surface configured to tamponade the hemostat, the applicator further including a pocket for receiving and storing the hemostat during delivery thereof.

In yet another embodiment, An instrument for delivery, deployment, and tamponade of a hemostat is provided and includes a delivery cannula having a proximal end, a distal end, and a lumen therebetween; a shaft having a distal end portion terminating at a distal end, and being configured to be received within the lumen and move relative to the lumen along an axis; and a hemostat applicator coupled to the distal end portion of the shaft, the applicator including a tamponade surface configured to be pressed against an area where the hemostat is deployed to apply pressure thereto. The distal end portion of the shaft is configured to articulate to a first angle relative to the axis.

In yet another embodiment, an instrument for delivery, deployment, and tamponade of a hemostat is provided and includes a delivery cannula having a proximal end, a distal end, and a first lumen therebetween; a shaft having a proximal end, a distal end, an outer wall between the proximal and distal ends, a second lumen between the proximal and distal ends, and an aperture traversing through the outer wall to the second lumen, the shaft being configured to be introduced into and move relative to the first lumen; and a hemostat configured to transition between a first, compact state for introduction into and movement within the first lumen, and a second, expanded state and further configured to be carried by the shaft regardless of whether the hemostat is in the compact or expanded state. The hemostat is positioned relative to the aperture to receive fluid when the second lumen is in fluid communication with a source of fluid.

In another embodiment of the invention, a method for delivery, deployment, and tamponade of a hemostat is provided and includes using a delivery shaft having a hemostat applicator coupled to a distal end portion of the delivery shaft, the hemostat applicator including a pocket for receiving and carrying the hemostat. The method includes inserting a delivery cannula having the delivery shaft therein into a trocar; moving the delivery shaft relative to the delivery cannula until at least the applicator exits from the delivery cannula, whereupon the applicator transitions from a first, compact state to a second, expanded state; and removing the hemostat from the pocket and deploying the hemostat to an area of tissue.

In another embodiment of the invention, a method of assembling an instrument configured for delivery, deployment, and tamponade of a hemostat is provided. The instrument includes a delivery cannula having a proximal end, a distal end, and a lumen therebetween, and delivery shaft having a proximal end, a distal end, and a hemostat applicator coupled to the distal end thereof, the hemostat applicator configured to transition between an expanded state and a contracted state and having a pocket for receiving the hemostat. The method includes directing a hemostat into the pocket and directing the proximal end of the delivery shaft into the distal end of the delivery cannula and further into the lumen until the applicator transitions from the expanded state to a contracted state and resides within the lumen.

In yet another embodiment of the invention, a method for delivery, deployment, and tamponade of a hemostat is provided. Using a delivery shaft having a hemostat applicator coupled to a distal end portion of the delivery shaft, the hemostat applicator including a tamponade surface, the method includes inserting a delivery cannula having the delivery shaft therein into a trocar; moving the delivery shaft relative to the delivery cannula until at least the applicator exits from the delivery cannula, whereupon the applicator transitions from a first, compact state to a second, expanded state; deploying a hemostat to an area of tissue; and tamponading the hemostat and the area of tissue using the applicator.

### BRIEF DESCRIPTION OF THE DRAWINGS

FIG. 1 is a perspective view of an instrument for delivering, deploying, and tamponading a hemostat, including a distal end portion being articulated and a hemostatic applicator thereof being in an extended position;

FIG. 2 is a detailed perspective view of a distal end of the instrument of FIG. 1;

FIG. 3 is a detailed perspective view of a distal end of the instrument of FIG. 1, showing a hemostat being directed into a pocket of a hemostatic applicator;

FIG. 4 is another detailed perspective view of a distal end of the instrument of FIG. 1;

FIG. 5 is a detailed perspective view of a distal end of the instrument, showing the hemostatic applicator being retracted into a delivery cannula and in an intermediate position;

FIG. 6 is a perspective view of the instrument of FIG. 1, shown in a ready position;

FIG. 7A is a detailed side cross-sectional view of the instrument of FIG. 1 showing the applicator retracted into a delivery cannula of the instrument and in a contracted position, taken along line 7-7 of FIG. 6;

FIG. 7B is a detailed side cross-sectional view of the instrument of FIG. 1 showing internal components, taken along line 7-7 of FIG. 6;

FIG. 8 is a detailed rear cross-sectional view of the instrument of FIG. 1 showing internal components, taken along line 8-8 of FIG. 6;

FIG. 8A is another detailed rear cross-sectional view of the instrument of FIG. 1 showing internal components, taken along line 8-8 of FIG. 6;

FIG. 9A is a detailed side cross-sectional view of the instrument of FIG. 1, with the hemostatic applicator in the extended position and in a straight configuration.

FIG. 9B is a detailed side cross-sectional view of the instrument of FIG. 1, with the hemostatic applicator in the extended position and in an articulated configuration;

FIG. 10 is a perspective view of the instrument of FIG. 1, showing the hemostat being removed from a pocket of the hemostat applicator; and

FIG. 11 is a perspective view of the instrument of FIG. 1 being used to tamponade the hemostat and target area.

FIG. 12 is a disassembled perspective view of an alternative embodiment of an instrument for delivering, deploying, and tamponading a hemostat.

FIG. 13 is an assembled perspective view of an alternative embodiment of the instrument of FIG. 12.

FIG. 14A is a partial side cross-sectional view of the assembled instrument of FIG. 13, taken along line 14-14, showing the applicator in a contracted state.

FIG. 14B is a partial side cross-sectional view of the assembled instrument of FIG. 13, taken along line 14-14, showing the applicator in an expanded state.

FIG. 14C is a cross-sectional view taken along line 14C-14C shown in FIG. 14B.

FIG. 15A is a front cross-sectional view of the assembled instrument of FIG. 13, taken along line 15-15, showing the applicator in a contracted state.

FIG. 15B is a front cross-sectional view of the assembled instrument of FIG. 13, taken along line 16-16, showing the applicator in an expanded state.

FIG. 16A is a partial side cross-sectional view of the assembled instrument of FIG. 13, taken along line 14-14, showing the applicator in a contracted state and fluid being directed out of a stylet.

FIG. 16B is a partial side cross-sectional view of the assembled instrument of FIG. 13, taken along line 14-14, showing the applicator in an expanded state and fluid impregnating at least the hemostat.

FIG. 16C is a cross-sectional view taken along line 14C-14C shown in FIG. 16B, showing fluid impregnating at least the hemostat

FIG. 17 is a front view of an alternative embodiment of an applicator.

FIG. 18A shows a hemostat being removed from the applicator of the instrument of FIG. 1.

FIG. 18B shows the applicator of FIG. 18A being used to tamponade the hemostat to a target area of tissue.

FIG. 19 is a front view of another alternative embodiment of an applicator.

FIGS. 20A and 20B are side views of a portion of the applicator in order to show certain properties thereof.

FIG. 21A is a front view of an alternative embodiment of a portion of an applicator.

FIG. 21B is a cross-sectional view of the portion of the applicator of FIG. 21A, taken along line 21B-21B.

FIG. 21C is a detailed cross-sectional view of the portion of the applicator of FIG. 21A, taken along line 21B-21B.

FIG. 22 is a disassembled perspective view of an alternative embodiment of an instrument for delivering, deploying, and tamponading a hemostat.

FIG. 23A is a side cross-sectional view showing one step of the instrument being assembled.

FIG. 23B is a side cross-sectional view showing the instrument assembled and in the ready position.

FIG. 23C shows the instrument being used to deliver and deploy the hemostat to a target area of tissue.

FIG. 23D shows the instrument being used to tamponade the hemostat to the target area of tissue.

FIG. 23E shows a portion of the instrument being retracted after being used to tamponade the hemostat.

### DETAILED DESCRIPTION

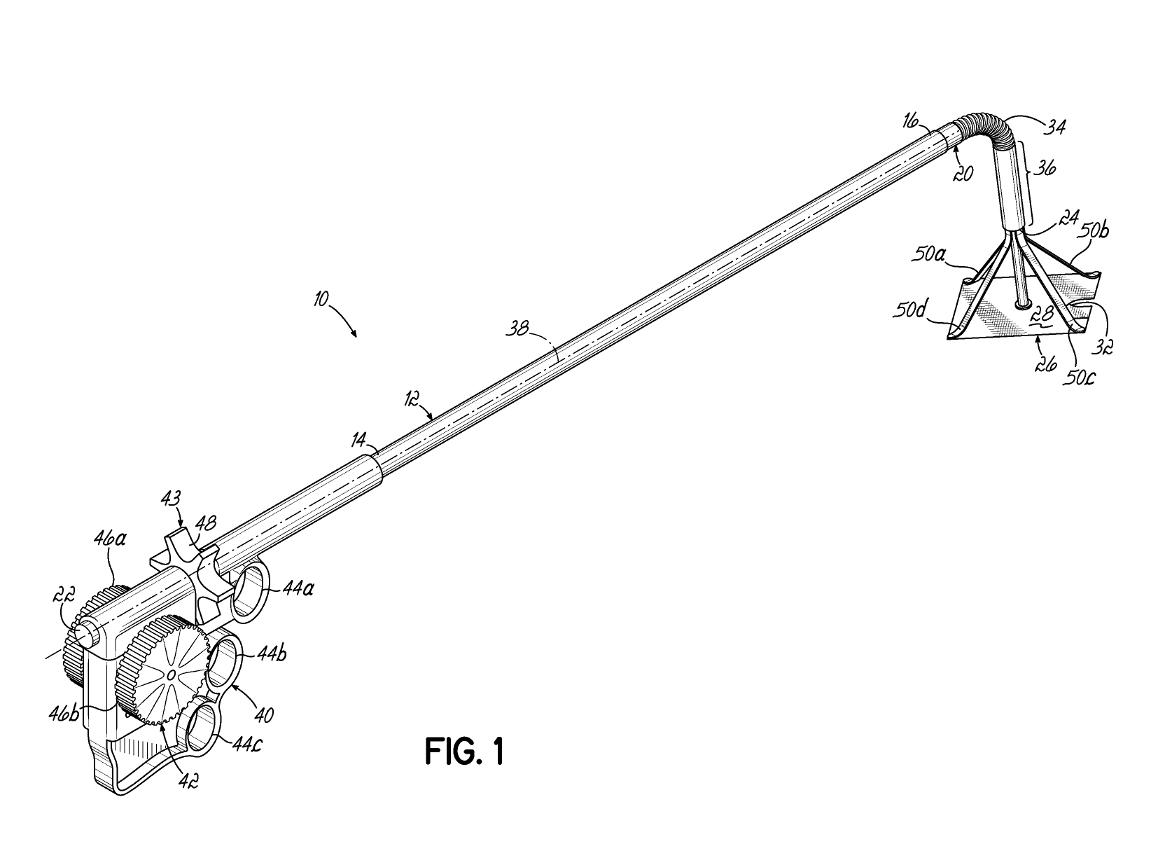

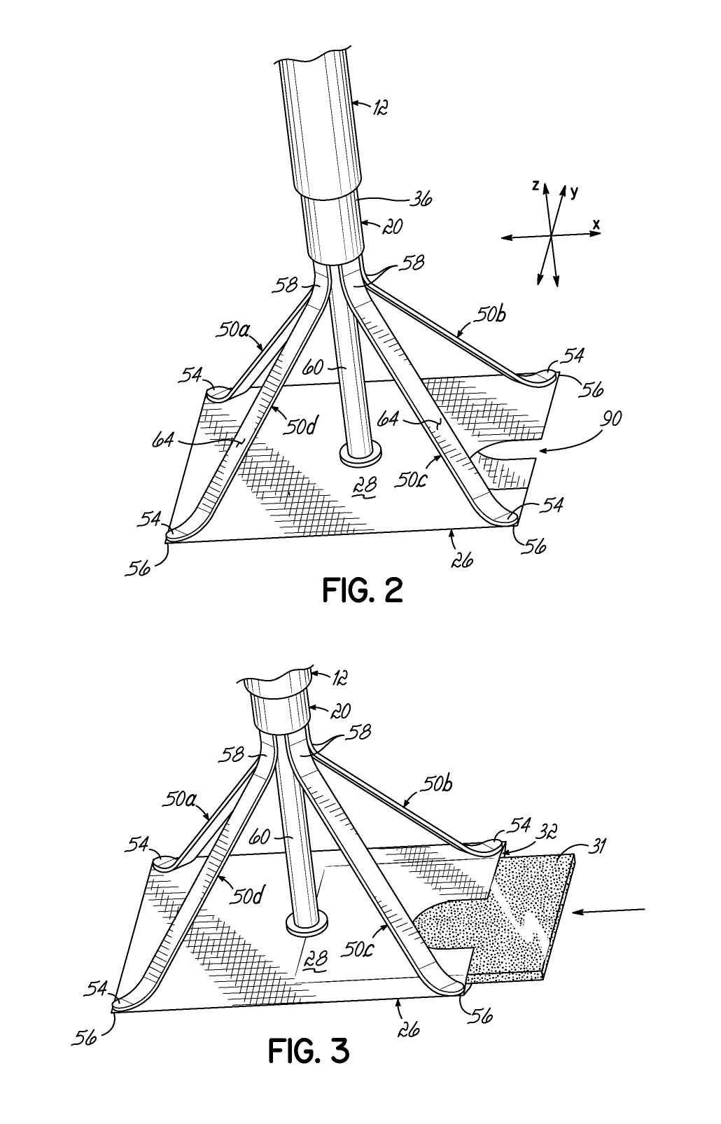

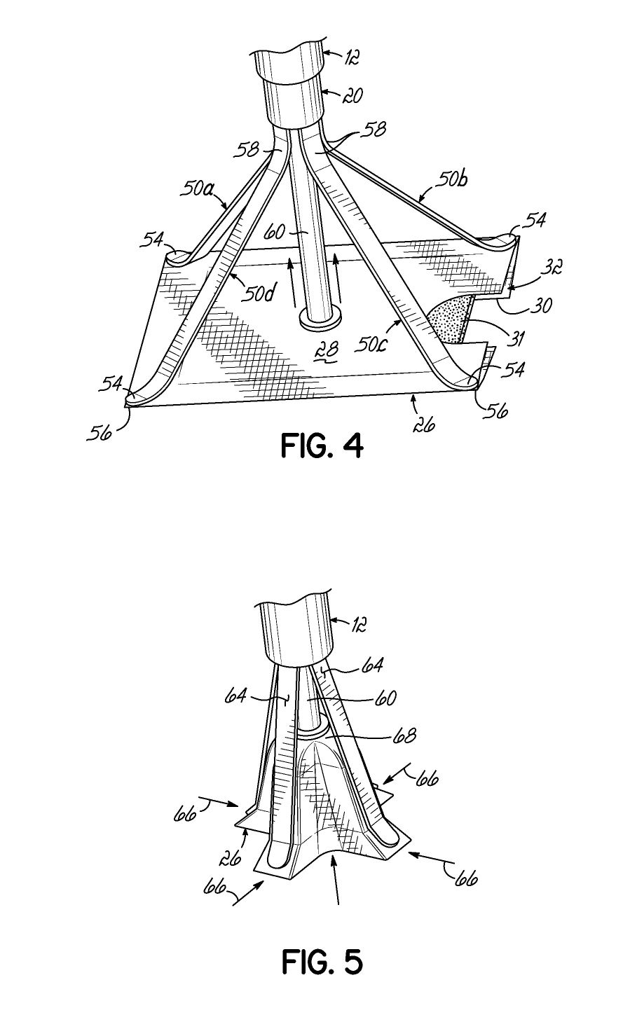

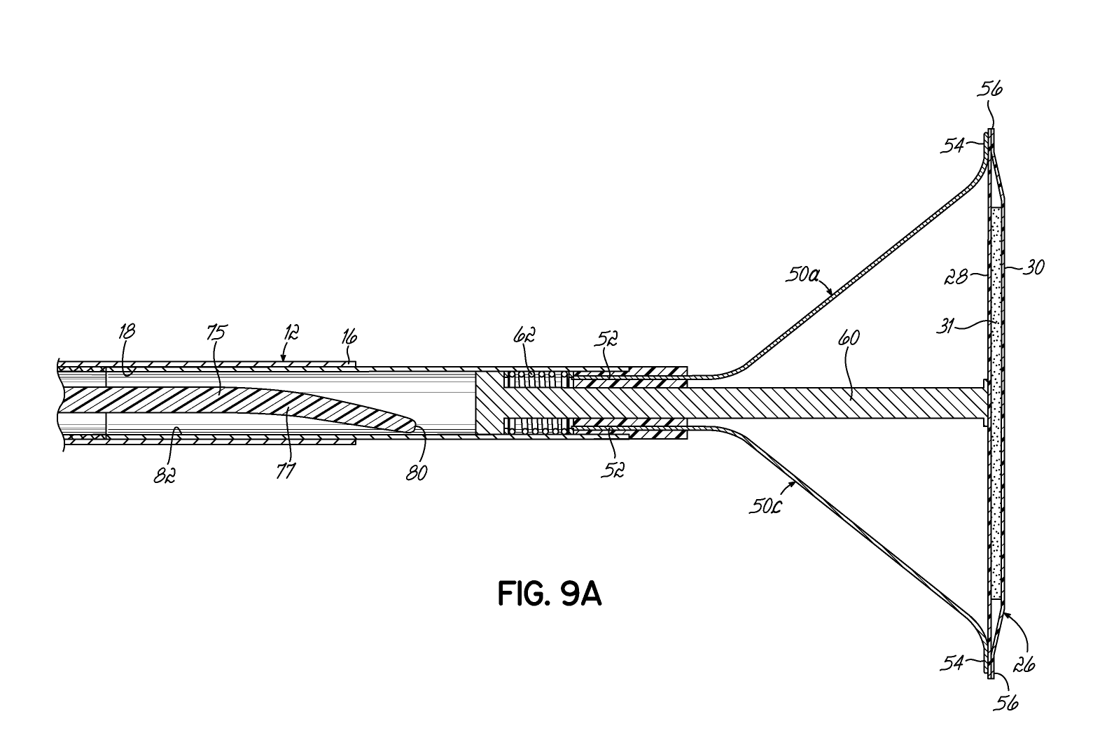

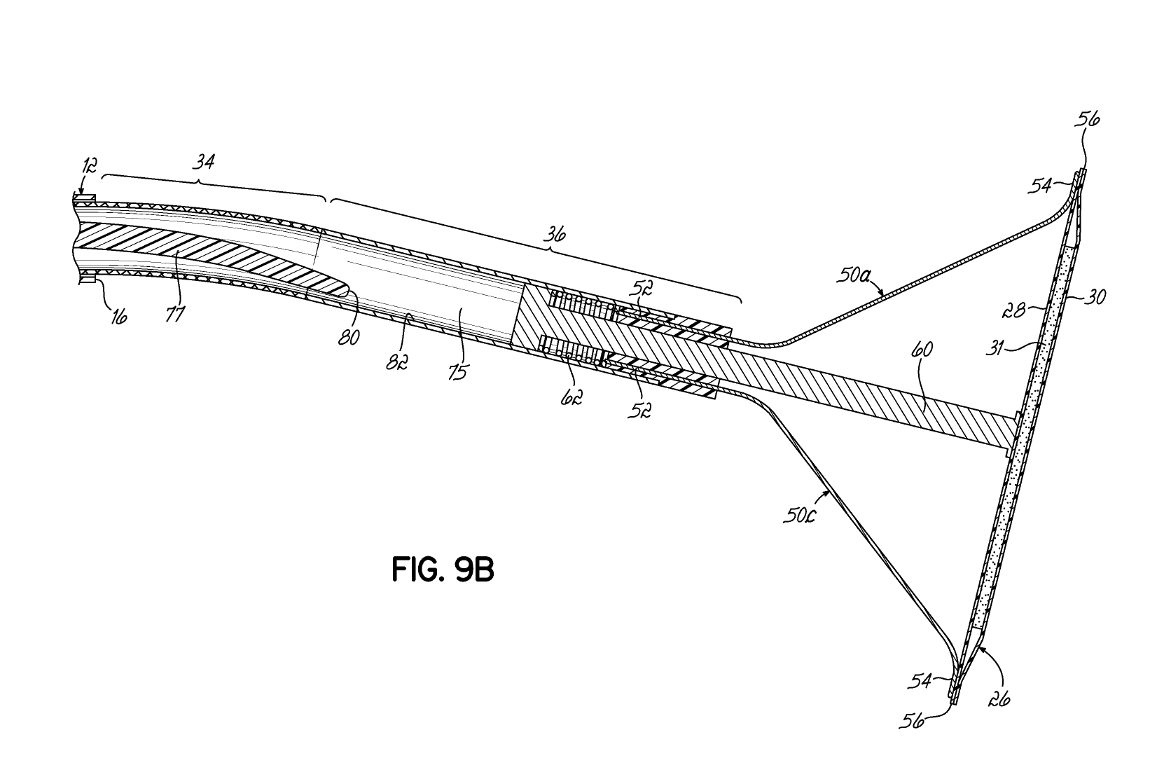

Referring to FIGS. 1-6, an instrument 10 according to one embodiment of the invention is shown. The instrument 10 includes an outer delivery cannula 12 having a proximal end 14, a distal end 16, and a lumen 18 extending between the proximal and distal ends 14, 16. An inner, delivery shaft 20 is shown received in the lumen 18 of the delivery cannula 12 such that the shaft 20 is movable relative to the delivery cannula 12. The shaft 20 includes a proximal end 22 and a distal end 24. The distal end 24 of the shaft 20 is coupled to a hemostat applicator 26 for applying hemostatic patches, also referred to as hemostats, to an area of tissue. The applicator 26 includes a proximal portion defined as a proximal face 28 and a distal portion defined as a distal face 30 (FIGS. 9A-B). The distal face 30 is configured to act as a tamponade surface for the hemostat 31 once the instrument 10 has been directed into a part of the body such as an insufflated abdomen. Thus, the distal face 30 may be considered a part or whole of the tamponade surface. The applicator 26 itself includes a pocket 32 between the proximal and distal faces 28, 30 for delivering the hemostat 31 (FIG. 3).

The shaft 20 includes a flexible portion 34 that is configured to bend as it exits from the distal end of the delivery cannula 12, thereby moving a distal end portion 36 of the shaft 20 at an angle relative to a longitudinal axis 38 of the instrument 10. Thus, the instrument 10 can be considered an articulating instrument such that a distal end portion 36 thereof is configured to move at an angle relative to, for example, a longitudinal axis 38 of the instrument 10.

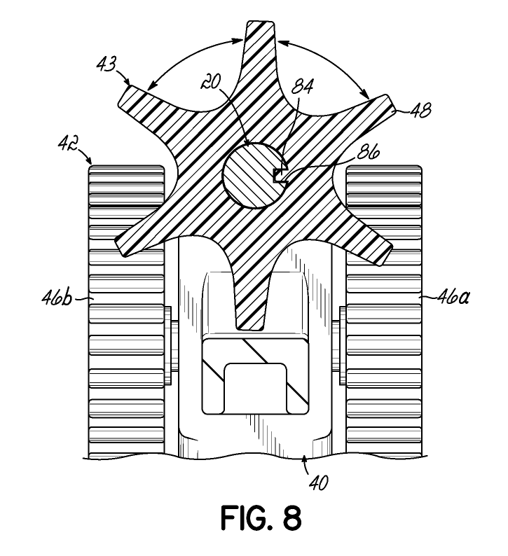

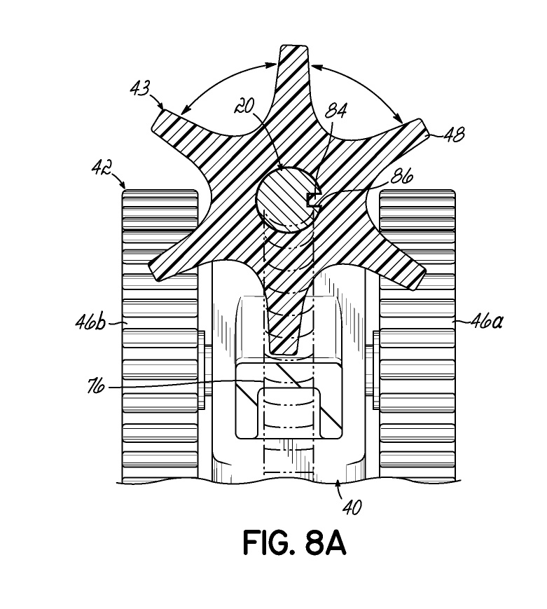

A handle assembly 40 includes a first actuator mechanism 42 that controls the axial position and movement of the shaft 20 relative to the delivery cannula 12. A second actuator mechanism 43 (see also FIG. 8) controls the rotational position of the shaft 20 relative to the delivery cannula 12. Because of the articulating nature of the distal end portion 36 of the shaft 20, and because the shaft 20 may rotate, the applicator 26 may be moved into any desired position in order to, for example, tamponade a hemostat 31 applied to a target area. Advantageously, the articulation of distal end portion 36 may allow the applicator 26 to reach several areas of the body without the need for additional laparoscopic or other minimally invasive incisions. As shown, the handle assembly 40 optionally includes finger holes 44a-c for ergonomic placement of a clinician's fingers during use. For example, a user may insert her index finger into the first hole 44a, her middle finger into the second hole 44b, and her ring finger in the third hole 44c, while using her thumb in order to actuate the left or right thumbwheel 46a, 46b of the first actuator mechanism 42 (depending on which hand is being used), or the thumbwheel 48 of second actuator mechanism 43. Of course, the configuration of the user or clinician's fingers relative to the finger holes 44a-c is not so limited. Furthermore, it will be appreciated that other configurations of the handle assembly 40 are possible.

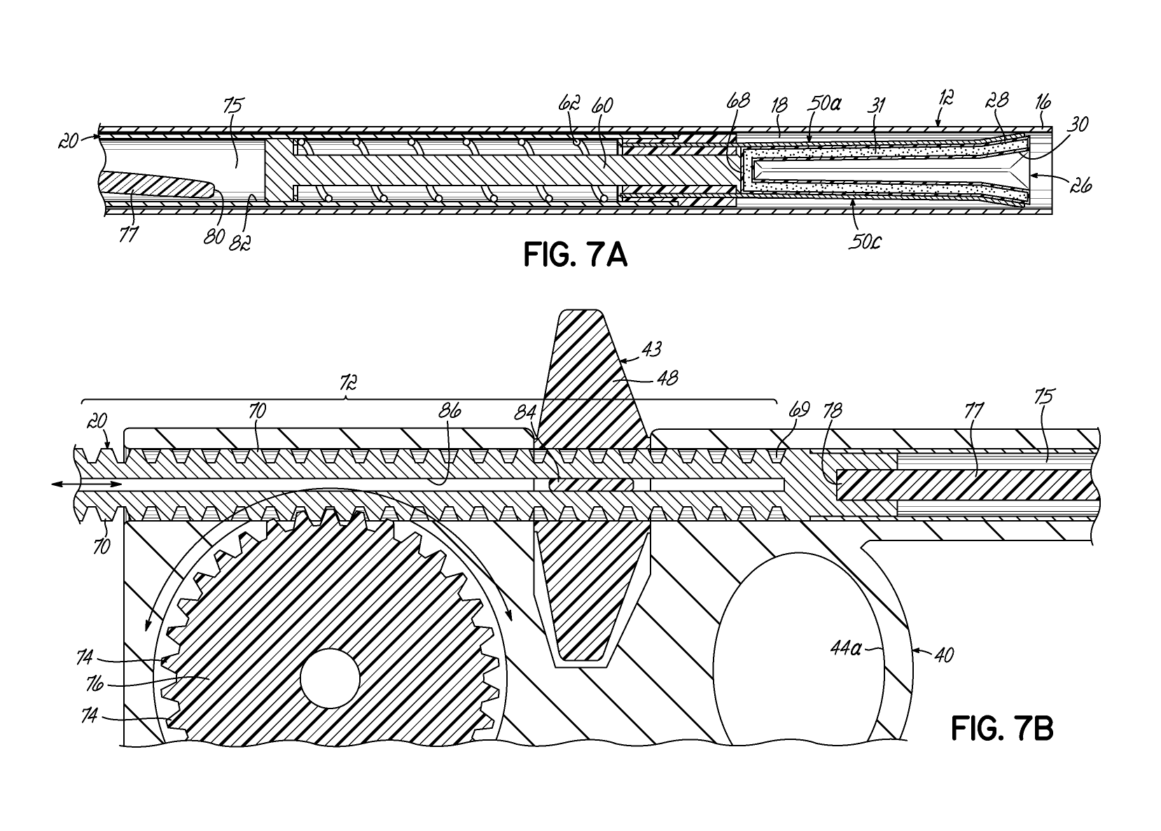

In order to prepare the instrument 10 for insertion into an endoscopic trocar (not shown) for delivery of the hemostat 31, the applicator 26 must contract into, or be collapsed into, a contracted state (FIG. 7A) from an expanded state such that it may reside in the delivery cannula 12. Normally, the applicator 26 is biased into the expanded state by resilient arms 50a-d due to each of the arms 50a-d flexing radially away from an axis along which the distal end portion 36 extends, which depends on whether and at what angle the distal end portion 36 is articulated, if at all. For example, if distal end portion 36 is not articulated, then it essentially extends along axis 38. Each arm 50a-d includes a first end 52 (FIGS. 9A-B) coupled to and extending distally from the distal end 24 of the shaft 20 and a second end 54 coupled to a respective corner portion 56 of the applicator 26. Arm 50a and arm 50c provide opposing forces along the x-y plane, while arms 50b and 50d similarly provide opposing forces along the x-y plane. As shown, the applicator 26 is a generally rectangular shape, but may alternatively be a different shape such as an alternative polygon or circle. In one embodiment where the applicator 26 is a polygon, the number of resilient arms may correspond to the number of corners or corner portions of the polygon. In one embodiment, the resilient arms 50a-d are spaced equally about a circumference defining at least a portion of the applicator (i.e., where the applicator is a regular polygon or a circle).

Each arm 50a-d includes a bent or resilient portion 58 that imparts at least a portion of the spring bias to the arms 50a-d. The spring bias may be imparted to the arms 50a-d due to pre-stressing, shape memory characteristics, or through other processes or characteristics that may give the arms 50a-d resilient or spring-like properties. Because of the opposing forces of opposing arms as described above, the applicator 26 is in a generally planar configuration in the expanded state. The proximal face 28 is coupled to a resilient pin 60 which is urged into and maintained in a distal position when the applicator 26 is in the expanded state. The force in the z direction on the resilient pin 60 provided by the resilient arms 50a-d, as transferred to the pin 60 from proximal face 28, is greater than the opposing force provided by compression spring 62 (FIGS. 7A, 9) on pin 60. Thus, with the applicator 26 in the expanded position, as best seen in FIG. 9, pin 60 is urged into a distal position by proximal face 28 of applicator 26, which thereby compresses spring 62.

Referring to FIG. 5, in order to collapse the applicator 26 into the contracted state, the shaft 20 is moved proximally relative to the delivery cannula 12. Once the proximal movement causes an outer surface 64 of the arms 50a-d to engage the distal end 16 of the delivery cannula 12, the arms 50a-d flex inwardly in the direction of arrows 66 and the force provided on the applicator 26 by the arms 50a-d decreases. Once this force, and specifically this force in the z direction, is less than the proximal biasing force of spring 62, spring 62 will move from its compressed state (FIG. 9) to its extended state (FIG. 7A) and thereby urge pin 60 in the proximal direction. As the pin 60 moves in the proximal direction, a center portion 68 of the proximal face 28 will also be moved proximally. The pin 60 therefore advantageously ensures that center portion 68 of proximal face 28 of applicator 26 is the first portion of the applicator 26 that enters the delivery cannula 12 as the shaft 20 is moved further proximally, such that the center portion 68 defines an apex of the applicator 26 in the contracted position (see FIG. 7A). Such a configuration also ensures that the applicator 26 is transitioned or moved to the contracted state within the delivery cannula 12 in a predictable manner, and also prevents unwanted contraction of the applicator 26. For example, resilient pin 60 prevents the corners 56 from folding to a contracted position such that corners 56 are situated proximately to the proximal face 28 (i.e., such that the applicator 26 folds oppositely as shown in FIG. 5). Notably, pin 60 and spring 62 may be configured such that the biasing force causes such folding or contraction, or to provide for other configurations of the applicator 26 in the contracted state. Ultimately, shaft 20 is moved in the proximal direction such that the applicator 26 and arms 50a-d reside within the lumen 18 of delivery cannula 12. Thus, a cross-sectional dimension of the applicator 26 in the expanded state is greater than the cross-sectional dimension in the contracted state.

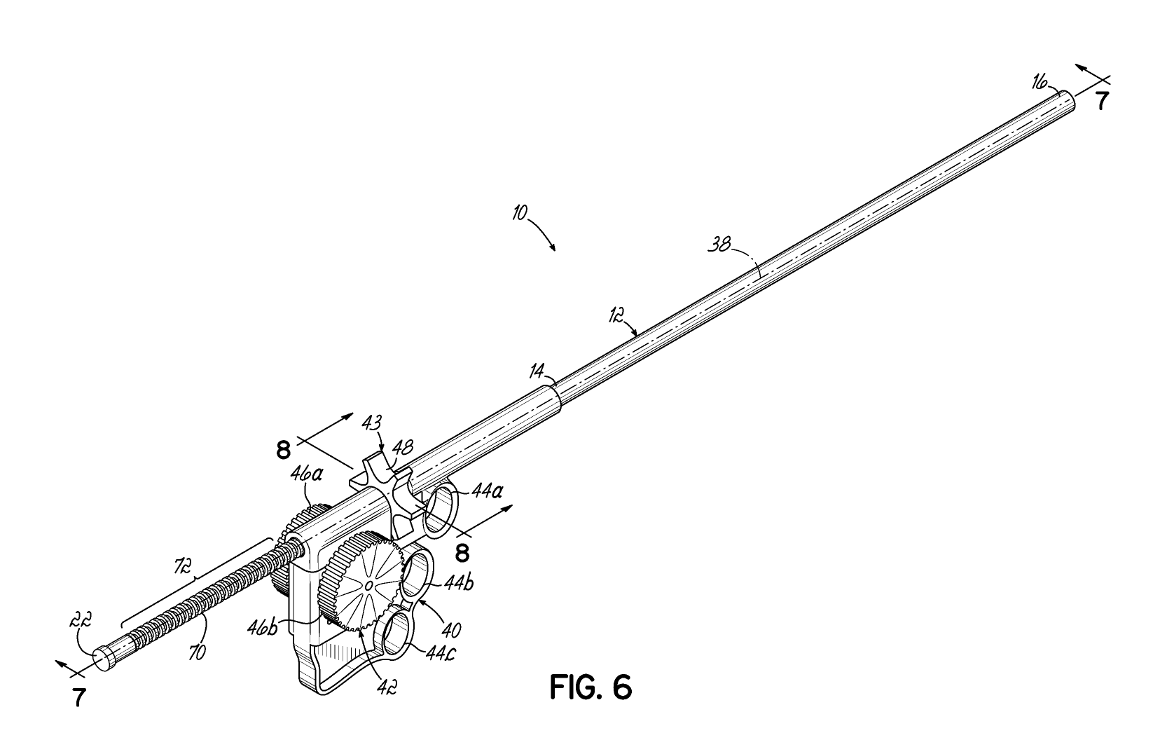

As discussed above, proximal movement of the shaft 20 relative to the delivery cannula 12 provides for retraction of the shaft 20 and applicator 26, as well as contraction of the applicator 26 within the delivery cannula 12. Initially, such proximal movement is simply due to the proximal end of shaft 20 being inserted into the distal end 14 of delivery cannula 12 and through lumen 18. Referring to FIG. 7B, the proximal end 22 of shaft 20 may then be moved further within the lumen 18 to a point where the shaft enters lumen 69 of the handle assembly 40, where a tooth or teeth 70 of a rack portion 72 coupled to (or a part of) shaft 20 mesh with a tooth or teeth 74 of a pinion gear 76 of the first actuator mechanism 42. The first actuator mechanism 42 may be considered to include at least the thumbwheels 46a, 46b, pinion gear 76, and rack portion 72. Alternatively, the first actuator mechanism 42 may be considered to include only thumbwheels 46a, 46b and pinion gear 76. Once meshed, either thumbwheel 46a, 46b may be rotated in the counter-clockwise direction (as viewed in FIG. 7B) to thereby rotate pinion gear 76 in the counterclockwise direction. Eventually, when the shaft 20 and applicator 26 reach the positions shown in FIG. 7A, the rack portion 72 of shaft 20 extends a length in the proximal direction from the handle assembly 40 as shown in FIG. 6. Such positions of the instrument 10 may be considered as “ready” positions such that the instrument 10 is ready for use such that it may be inserted into an endoscopic trocar (not shown) or other access device and into the body for delivery, deployment, and tamponade of a hemostat 31. Notably, the relative positions and lengths of the components are not limited to those shown.

Preferably, the instrument 10 is assembled and in the ready position, including a hemostat 31 placed in the pocket 32, prior to being packaged and sent to a clinical environment for use by a clinician. Alternatively, the instrument 10 may arrive to the clinical environment in the disassembled state and a clinician may assemble the instrument 10 into the ready position. Notably, the instrument 10 may be reused after being used to deliver a hemostat 31, by simply inserting another hemostat 31 into the pocket 32 and retracting the shaft 20 within the delivery cannula 12, as described herein, such that the instrument 10 is again in the ready position.

In order to deliver the hemostat 31 to a target area, such as bleeding tissue within an insufflated abdomen or other body cavity, a clinician gains access to the abdomen through an endoscopic trocar or other access device (not shown), as is well known in the art. Notably, while access to the body through a trocar is preferred, it is not required. The clinician then directs the distal end of the delivery cannula 12 into the trocar until the distal end of the delivery cannula 12 resides within the body. In order to advance the shaft 20 distally relative to the delivery cannula 12, one or both of the thumbwheels 46a, 46b is rotated in the clockwise direction (as viewed in FIG. 7B) to thereby rotate the pinion gear 76 in the clockwise direction. Rotation of the pinion gear 76 in the clockwise direction thereby advances rack portion 72 and thus shaft 20 in the distal direction. As the shaft 20 moves distally relative to the delivery cannula 12, the applicator 26 may transition from the first, contracted position to the second, expanded position, in the same but essentially reverse manner as during the transition from the expanded position to the contracted position as the shaft 20 and applicator 26 are retracted proximally into the delivery cannula 12 to put the instrument 10 in the ready position.

In that regard, as the second ends 54 of each of the arms 50a-d begin to exit and further protrude from distal end 16 of delivery cannula 12, arms 50a-d begin to flex outwardly away from axis 38 due to the outward bias imparted to arms 50a-d. As more of the length of arms 50a-d protrudes from the delivery cannula 12, the biasing force from each of the arms 50a-d urges a respective corner 56 of applicator 26 in the same direction. Therefore, while corners 56 of applicator 26 are urged outwardly and downwardly, center portion 68 of applicator 26 is thereby urged downwardly. The downward movement of center portion 68 is due to the overall force in the z direction due to the resilient arms 50a-d being greater than the force provided on pin 60 by spring 62, as discussed above. Therefore, an intermediate position of the applicator 26 as it moves from the contracted state to the expanded state is essentially the same as the intermediate position as the applicator 26 moves from the expanded state to the contracted state, as shown in FIG. 5. Further distal movement of shaft 20 relative to the distal cannula 12 allows the arms 50a-d to reach the position shown to thereby transition the applicator 26 to the expanded state. Notably, because the speed at which the arms 50a-d and applicator 26 expand outwardly is a function of the speed of axial movement of the shaft 20 relative to the delivery cannula 12, the clinician may allow expansion of the arms 50a-d and applicator 26 in a controlled manner. Thus, simple relative movement between the shaft 20 and the delivery cannula 12 allows the applicator 26 to be transitioned from the contracted to the expanded state, and vice versa.

Articulation of the distal end portion 36, in one embodiment, is effectuated by further distal movement of the shaft 20 relative to the delivery cannula 12. Referring to FIGS. 9A-B, shaft 20 includes lumen 75 having a resilient member 77 therein. Resilient member 77 is shown as a wire or a rod, but is not so limited such that, alternatively, it may be a hollow tube, a coiled structure, or the like. Resilient member 77 is fixed at a first end 78 (FIG. 7B) thereof to the rack portion 72 such that resilient member 77 moves with the axial movement of the rack portion 72 and shaft 20. In FIG. 7A, resilient member 77 is shown to be at least partially flexed such that a second end 80 thereof is bearing against an inner wall 82 of lumen 75 of shaft 20. In one embodiment, the resilient member 77 flexes or bends due to shape memory characteristics, but the flexing and bending may be due to other characteristics or processes, such as pre-stressing. In its normal position, at least a first portion of resilient member 77 extends along axis 38, and at one other portion of the member is bent or flexed relative to the first portion. Thus, placing the resilient member 77 in the lumen 75 of shaft 20 causes the second end 80 of resilient member 77 to bear against the inner wall 82 and impart a force on the inner wall 82.

The second end 80 of resilient member 77 extends from a proximal portion of the lumen 75, past a flexible portion 34 of the shaft 20, and terminates in the lumen 75 at a point adjacent to distal end portion 36 of shaft 20. Thus, the second end 80 of resilient member 77 bears against inner wall 82 of shaft lumen 75 at a point adjacent the distal end portion 36. Therefore, after distal end portion 36 has exited from the delivery cannula 12, and as flexible portion 34 also begins to exit from delivery cannula 12, flexible portion 34 is able to flex or bend under the force of resilient member 77 on inner wall 82 and thereby moves the distal end portion 36 at a first angle relative to axis 38. In one embodiment, the resilient member 77 is configured such that the further the length of resilient member 77 that extends past the distal end 16 of delivery cannula 12, the further the flexible member is able to bend to thereby increase the first angle. Of course, the maximum angle at which the distal end portion 36 will bend is limited by the curvature and bending force from the resilient member 77 as well as the resistance to flexing provided at the flexible portion 34. For example, where the flexible portion 34 includes coils (not shown), the interaction between the coils as the flexible portion 34 bends only allows a certain amount of bending. Notably, extending the shaft 20 to a point where the shaft 20 begins to articulate is optional and may not be necessary depending on the location on the anatomy where the hemostat 31 must be placed and the difficulty of reaching such a position.

If needed or desired, the shaft 20 may be rotated relative to the delivery cannula 12 about axis 38, thus rotating applicator 26 about axis 38 as well. Rotation of shaft 20 is effectuated by rotating the wheel 48 (FIG. 1). With reference to FIGS. 7B and 8, thumbwheel 43 includes a keyed portion 84 that interacts with a key slot 86 in shaft 20 such that rotation of thumbwheel 43 thereby rotates shaft 20. Referring to FIG. 8A, teeth 74 of pinion gear 76 (shown in phantom) are contoured to match the radially defined teeth 70 of rack portion 72. The contoured configuration of pinion gear 76 prevents the keyed portion 84 from interfering with smooth engagement of teeth 74 of pinion gear 76 and the teeth 70 of rack portion 72 during rotation of shaft 20 (due to rotation of thumbwheel 43). Smooth engagement between teeth 74 and teeth 70 is particularly advantageous during simultaneous rotation of thumbwheel 43 (to rotate shaft 20 about axis 38) and rotation of either thumbwheel 46a, 46b to move shaft axially along axis 38. As shown, the contour of pinion gear 76 is essentially concave but, in an alternative embodiment, the contour could be convexly shaped, depending on the corresponding contoured shape of the rack portion 72.

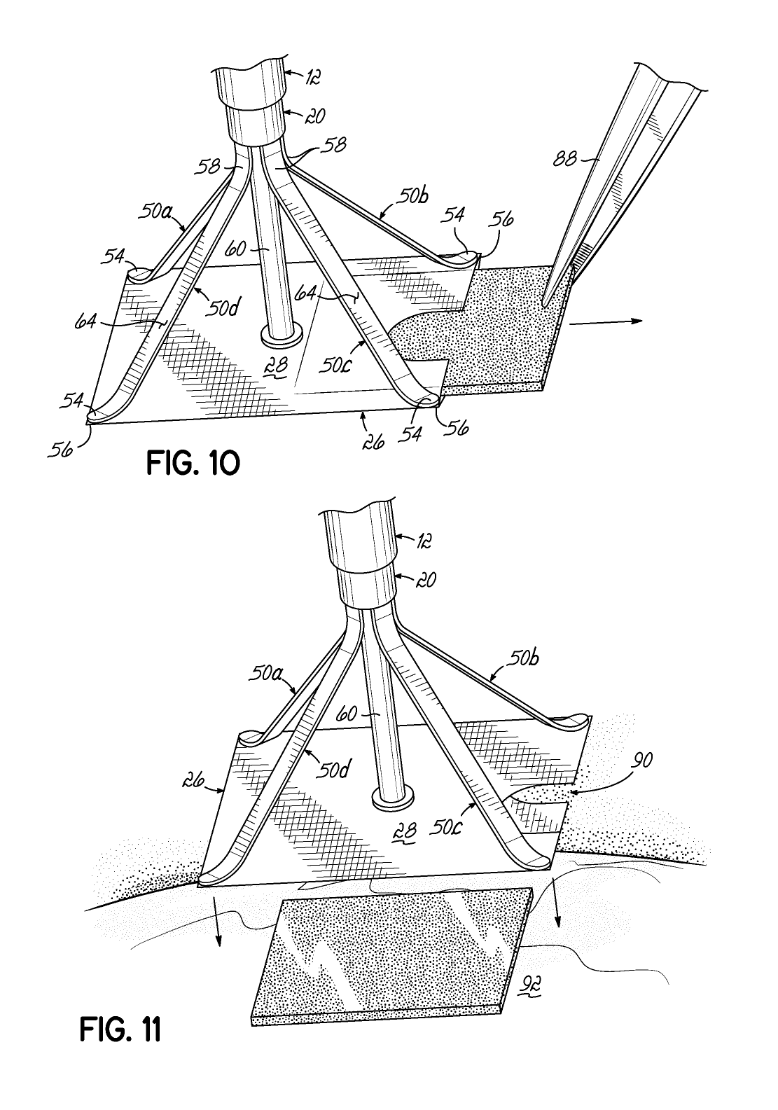

Once the applicator 26 is in the extended position, and regardless of whether the shaft 20 is articulated or rotated relative to delivery cannula 12, the hemostat 31 may be removed from the pocket 32 using surgical graspers 88 or other like instrument. Notably, the pocket 32 includes an aperture 90 on each of distal and proximal faces 28, 30 that provides a space for the graspers 88 to grasp hemostat 31. Rather than risking incorrect or misplaced deployment of the hemostat 31, the pocket 32 provides a storage space for the hemostat 31 that provides a moisture barrier in the collapsed and extended positions, and also allows the clinician to grasp the hemostat 31 with a graspers 88 (or other instruments) (FIG. 10). Once the clinician grabs the hemostat 31, it may be placed on or deployed to a target area 92, such as a portion of the body (i.e. an organ) that is bleeding. Then, the distal face 30 of applicator 26 may be used as a tamponade surface to apply pressure to the hemostat 31 (FIG. 11) and the target area 92 for a period of time until, for example, the target area 92 has stopped bleeding. At this point, the shaft 20 may be retracted into the delivery cannula 12 as described herein and the instrument 10 then can be removed from the trocar. Then, if desired, another hemostat 31 may be directed into the pocket 32, and the instrument 10 may be inserted into the trocar again for delivery of the hemostat 31, once the instrument 10 is in the ready position.

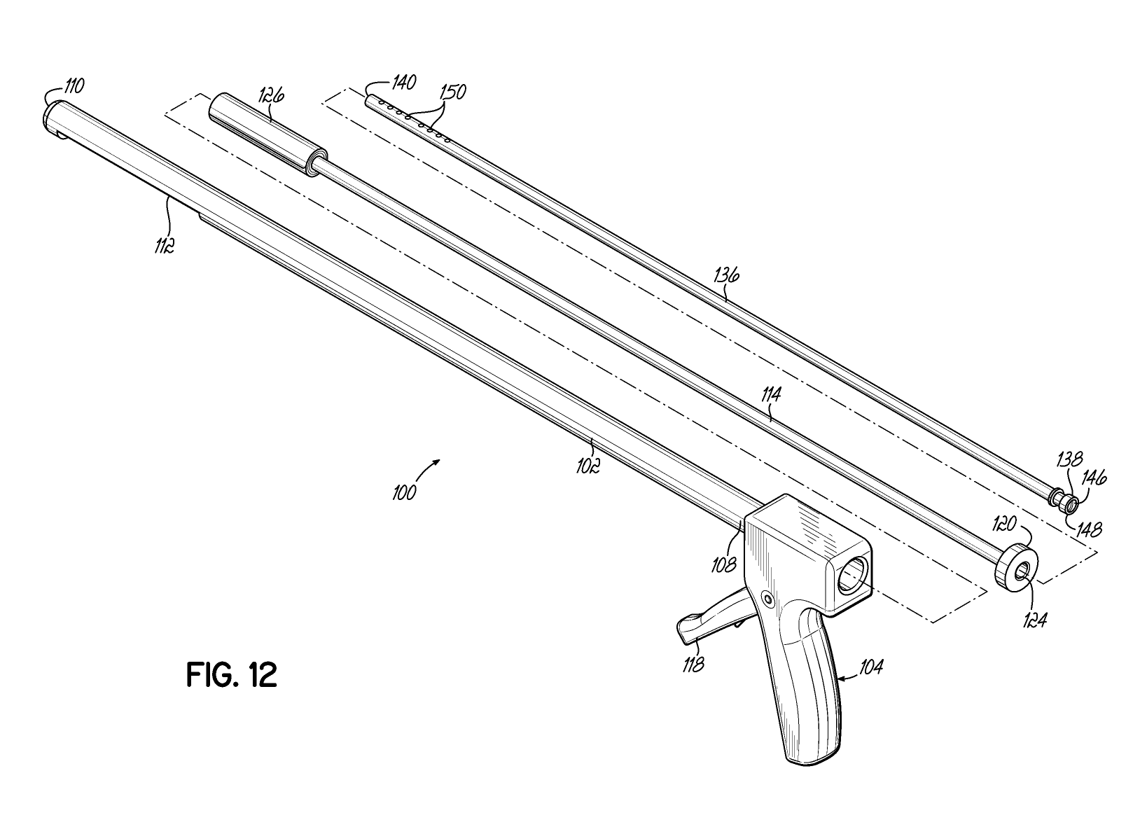

Referring to FIGS. 12-15B, another embodiment of an instrument 100 is shown. The instrument 100 includes a delivery cannula 102 having an optional handle and trigger mechanism 104 for selectively advancing other components of the instrument 100 within the lumen 106 of the delivery cannula 102. The lumen 106 extends between proximal and distal ends 108, 110 of the delivery cannula 102. A distal end portion includes an elongate aperture 112, the purposes of which are discussed in more detail below.

The instrument 100 also includes a shaft 114 (also referred to herein as “delivery shaft”) that is configured to carry or deliver a hemostat 116 through the delivery cannula 102 to thereby deploy and subsequently tamponade the hemostat and a target area of tissue. The shaft 114 may move relative to the delivery cannula 102 by simple manual movement of the shaft 114. Alternatively, the handle and trigger mechanism 104 may incrementally advance the shaft 114 within the delivery cannula 102 upon squeezing the trigger 118.

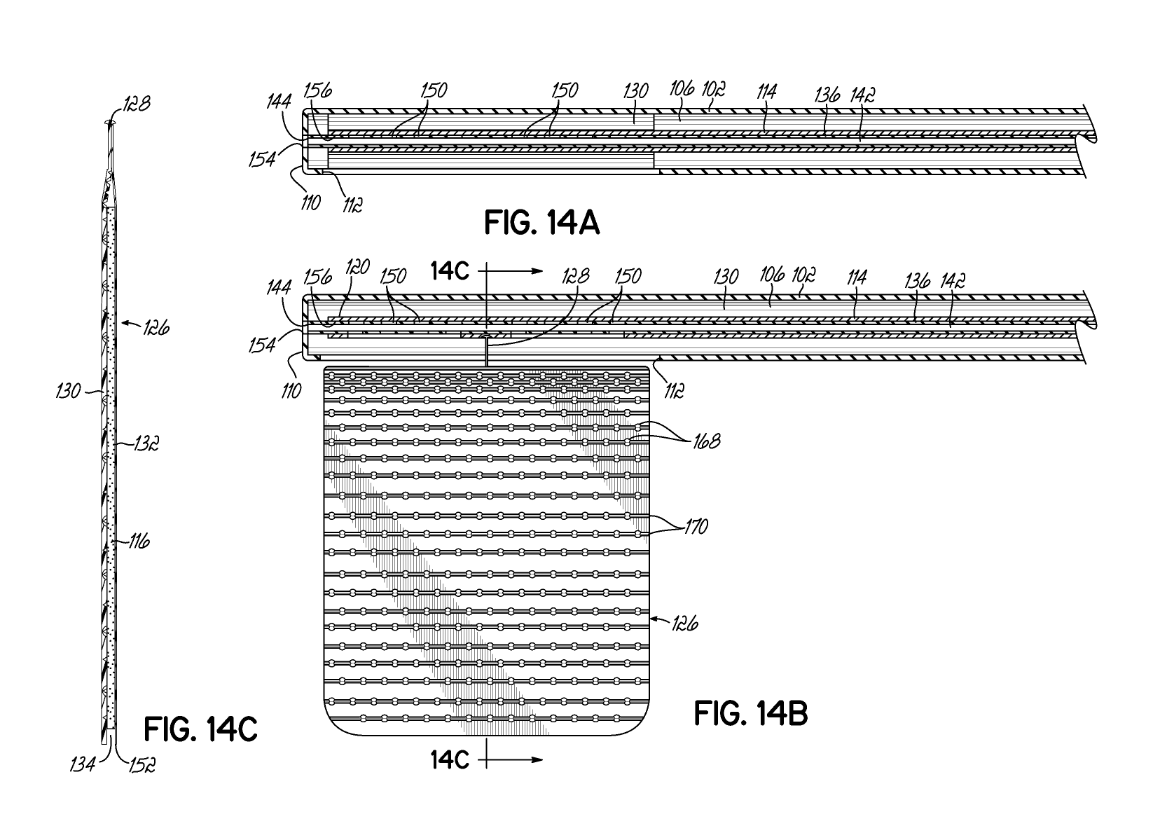

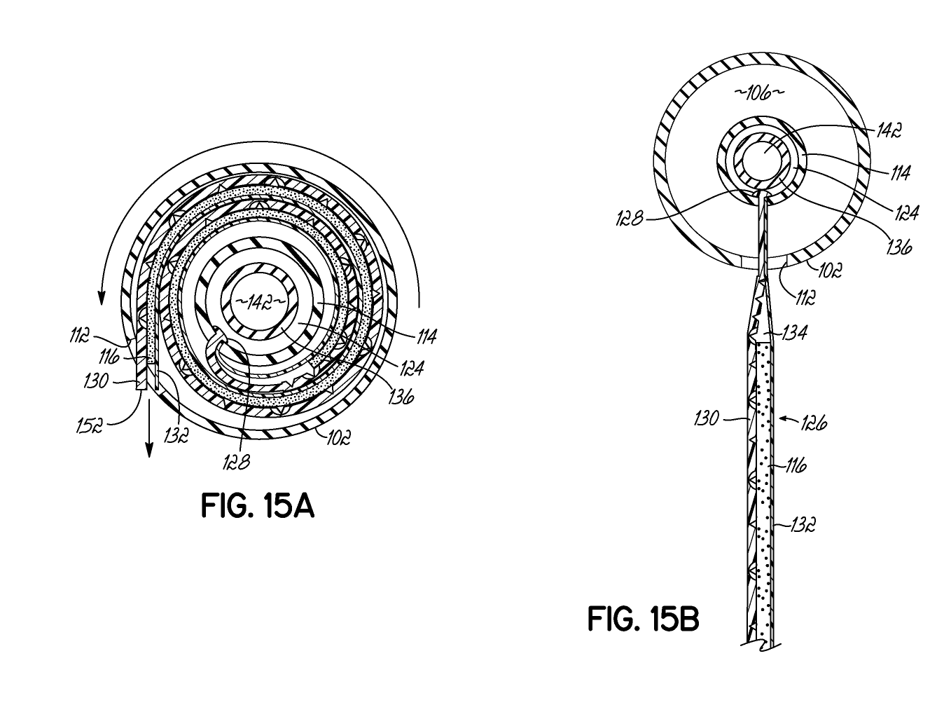

The shaft 114 includes a proximal end 120, a distal end 122, and a lumen 124 therebetween. At or near the distal end 122 of the shaft 114, an applicator 126 for holding or carrying a hemostat 116 is coupled to the shaft 114. The applicator 126 is hingedly connected to the shaft 114 by a pin 128 such that the applicator 126 may rotate relative to the shaft 114 about an axis that is perpendicular to an axis of the shaft 114. Alternatively, the applicator 126 may be overmolded onto the shaft 114. The applicator 126 includes a first portion 130 (or first side) and a second portion 132 (or second side). Each of the first and second portions 130, 132 is further defined as a flag-like structure. The first and second portions 130, 132 may include the same or different properties, sizes, materials, etc., as one another, as discussed below. There is a space or pocket 134 defined between the first and second portions 130, 132 for holding or carrying the hemostat 116. The applicator 126 is shown in a first, compact state, which allows for introduction of the applicator 126 (and the shaft 114) into, and movement within, the lumen 106 of the delivery cannula 102. In the compact state, the applicator 126 is in a rolled-up configuration. As discussed below, the applicator 126 may transition to a second, expanded state (FIGS. 14B and 15B) as the applicator 126 exits from the delivery cannula 102.

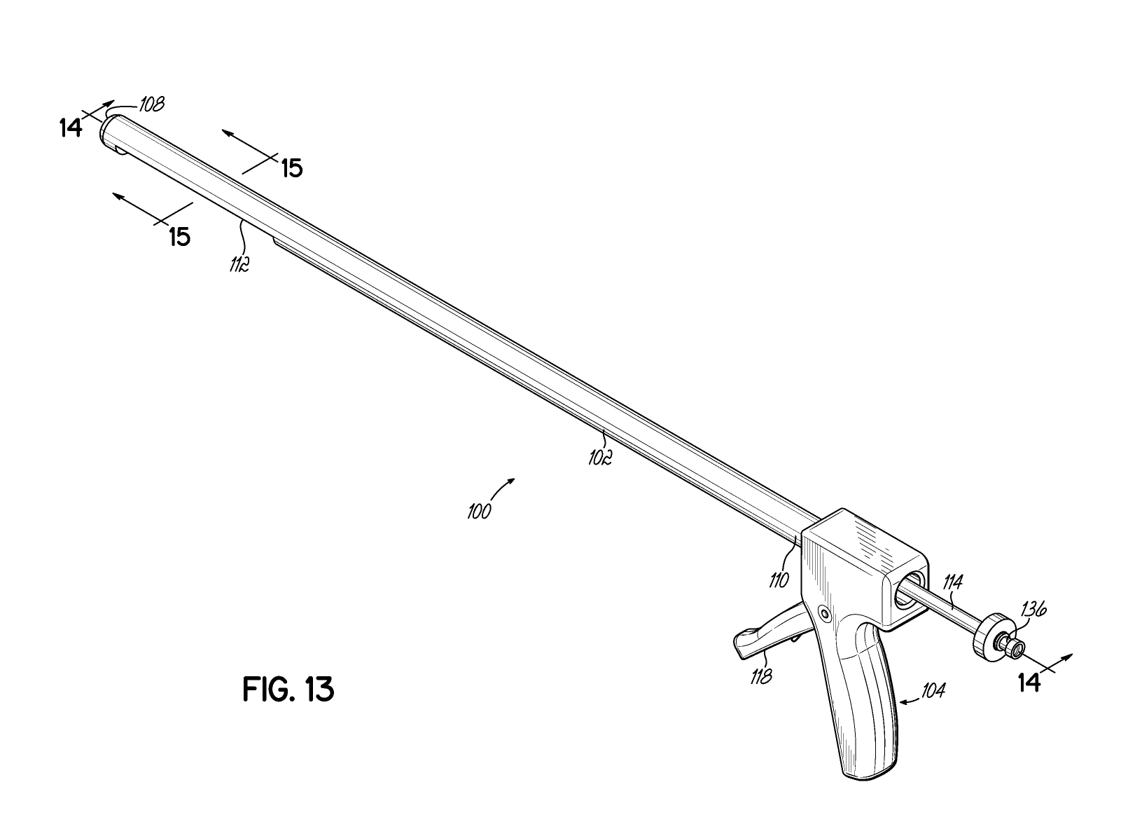

The instrument 100 also includes a stylet 136 having a proximal end 138, a distal end 140, and a lumen 142 therebetween. The stylet 136 is inserted into the lumen 124 of the shaft 114. The stylet 136 includes an opening 144 (FIG. 14A) at the distal end 140 and an opening 146 having a luer fitting 148 at the proximal end 138, as well as a plurality of apertures 150 near the distal end 140, for reasons that will become clear with reference to the discussion below. The instrument 100 is shown assembled in FIG. 13 with the shaft 114 inserted into the lumen 106 of the delivery cannula 102 and the stylet 136 inserted into the lumen 124 of the shaft 114.

In order to assemble the instrument 100 into a ready position (FIGS. 13, 14A, 15A), a hemostat 116 is preferably inserted into the pocket 134 of the applicator 126. The applicator 126 is then rolled up into the compact configuration so that the shaft 114 may be directed into the lumen 106 of delivery cannula 102. As before, the handle and trigger mechanism 104 may be used in order to distally move the shaft 114 relative to the delivery cannula 102. The stylet 136 may then be directed into the lumen 124 of shaft 114. However, the stylet 136 may be inserted into lumen 124 of shaft 114 before shaft 114 is directed into the delivery cannula 102.

Preferably, the instrument 100 is assembled and in the ready position, including a hemostat 116 placed in the pocket 134, prior to being packaged and sent to a clinical environment for use by a clinician. Alternatively, the instrument 100 may arrive to the clinical environment in the disassembled state and a clinician may assemble the instrument 100 into the ready position. Notably, the instrument 100 may be reused after being used to deliver a hemostat 116, by simply inserting another hemostat 116 into the pocket 134 and retracting the shaft 114 within the delivery cannula 102, as described herein, such that the instrument 100 is again in the ready position.

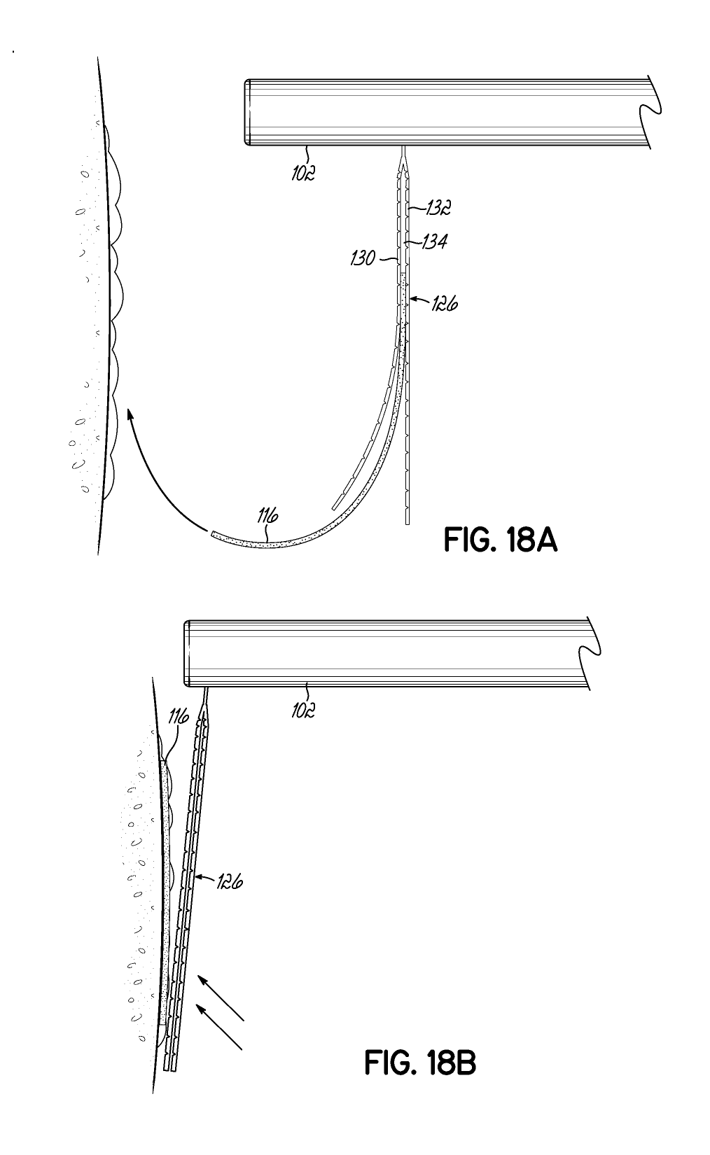

In order to deliver the hemostat 116 to a target area, such as bleeding tissue within an insufflated abdomen or other body cavity, a clinician gains access to the abdomen (or other part of the body) through an endoscopic trocar or other access device (not shown), as is well known in the art. Notably, while access to the body through a trocar is preferred, it is not required. The clinician then directs the distal end 110 of the delivery cannula 102 into the trocar until the distal end 110 of the delivery cannula 102 resides within the body. In order for the applicator 126 to transition to the expanded state, the shaft 114 is rotated until the bottom end 152 of the applicator 126 exits from the elongate aperture 112. The clinician then continues to rotate the shaft 114 such that the remaining portion of the applicator 126 exits from the delivery cannula 102. At this point, the clinician may remove the hemostat 116 from the pocket 134 of the applicator 126 (FIG. 18A) and apply it to a target area of tissue, and proceed to tamponade the hemostat 116 using the applicator 126 (FIG. 18B).

However, before deploying the hemostat 116, it may be desirable to clear the target area of blood, tissue, or debris using air or another fluid. Moreover, it may be desirable to apply a liquid, powder, or gel spray-on adhesive barrier, a coagulant such as fibrin or platelet rich plasma, or other materials such as a hydrogel (i.e., for coagulation or other purposes). If so, the clinician may fluidicly couple or communicate a source of air, liquid, or powder (not shown) with the stylet 136 so that the desired material is directed into the proximal end opening 144, travels through the lumen 142, and out of distal end opening 146.

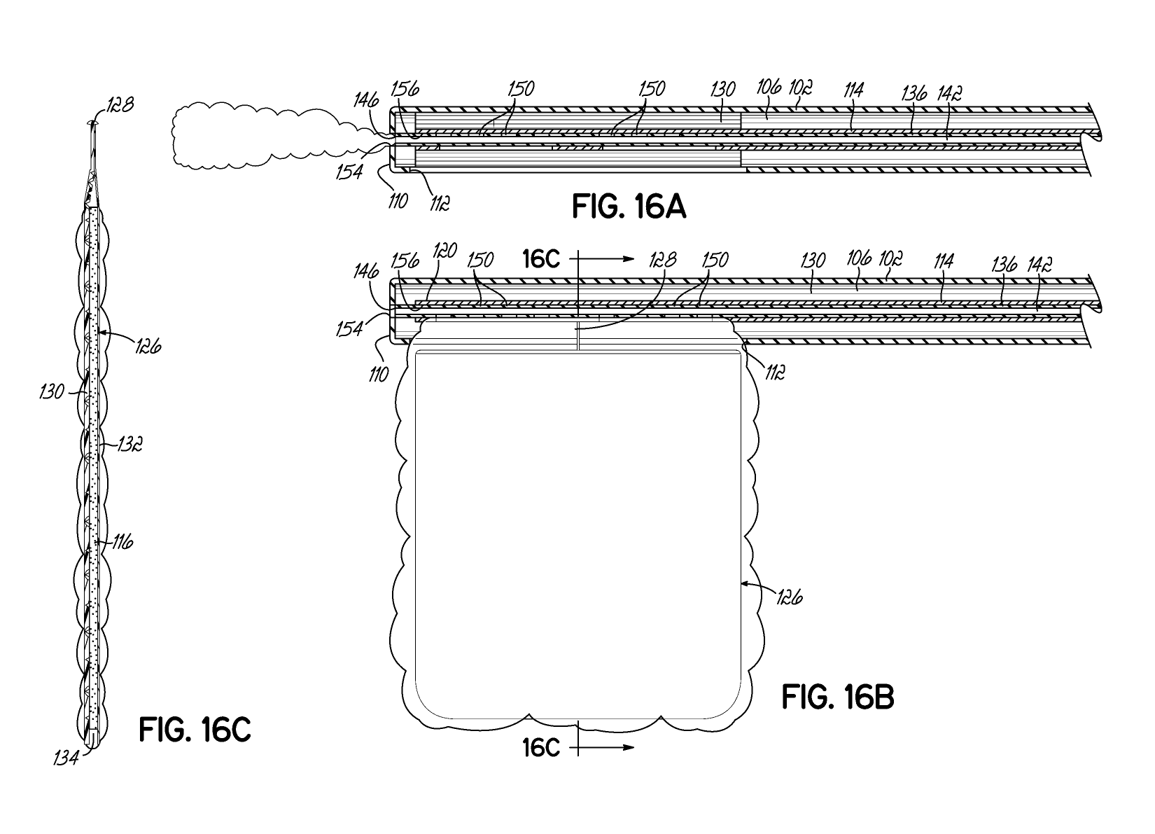

Because the apertures 150 are provided on one side of the stylet 136, the rotational position of the stylet 136 may be chosen to cause the air, liquid, or powder to exit from the opening of stylet 136 rather than the apertures 150. In that regard, stylet 136 is rotated to a position shown in FIG. 16A such that the air, material, or other substance is directed out of the opening of stylet 136 rather than out of the apertures 150. In other words, the air, material, or other substance will exit from the opening rather than the apertures 150 because the apertures 150 are at least partially blocked by the inner wall of the shaft 114, thus increasing resistance against the fluid leaving the apertures 150. In such a position, the apertures 150 preferably face away from the elongate aperture 112 of delivery cannula 102, such that apertures 150 are spaced 180 degrees from the elongate aperture 112. Similarly, the apertures 150 may be spaced 180 degrees from the hinge pin 128 which connects shaft 114 and applicator 126. Furthermore, in order to facilitate such a use of air, distal end of delivery cannula 102 includes an opening 154 and distal end of shaft 114 also includes an opening 156. While opening 154 of delivery cannula 102 is only shown to be a portion of distal end, the opening 154 may be larger or smaller than the size shown. For example, the opening 154 may be large enough for stylet 136 to travel out of the delivery cannula 102.

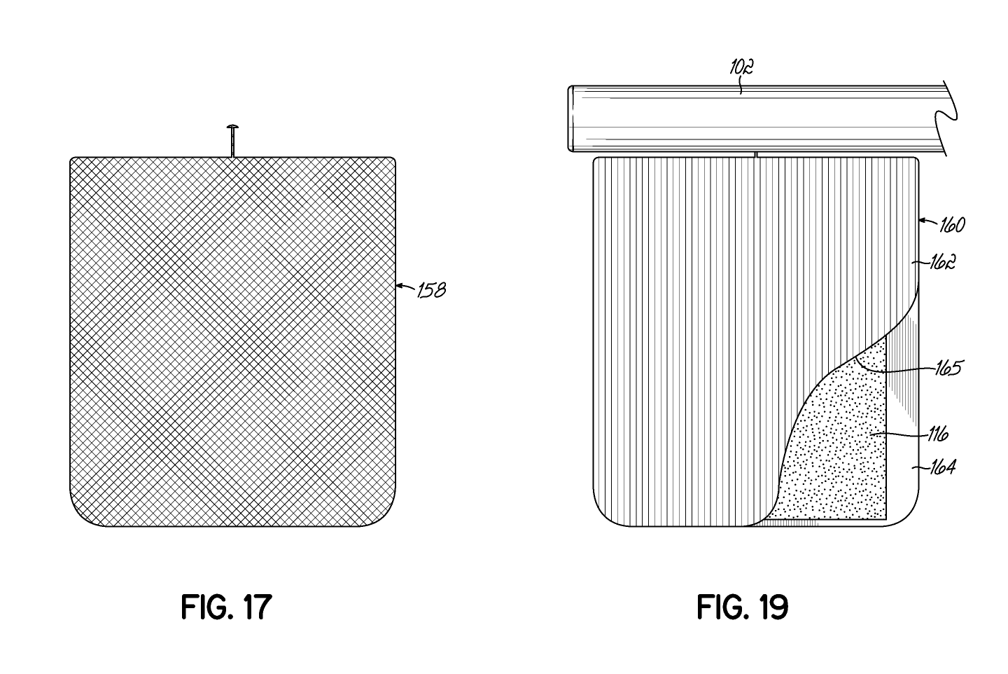

It may also be desirable to impregnate the hemostat 116 with a liquid, such as saline, growth hormones, or other liquids that may provide for improved hemostatic or other desired properties. To that end, stylet 136 is configured to receive a liquid and includes apertures 150, as disclosed above. When apertures 150 are properly positioned relative to the shaft 114 and the delivery cannula 102, the liquid will flow from apertures 150, and onto (or into) applicator 126 and impregnate the hemostat 116, shown in FIGS. 16B-C. In one embodiment, fluid is directed into the stylet 136 when the applicator 126 is in the expanded, unrolled position such that the fluid may travel between the first and second portions 130, 132 of the applicator 126 and impregnate the hemostat 116. In embodiments where the first and second portions 130, 132 of the applicator 126 are liquid impermeable, the clinician would rely on the fluid traveling between the first and second portions 130, 132 when the applicator 126 is in the expanded position, in order for the fluid to impregnate the hemostat 116. However, when one or both of the first or second portions 130, 132 of the applicator 126 are liquid permeable, such as the embodiment of applicator 158 shown in FIG. 17, the hemostat 116 could be impregnated with fluid when the applicator 158 is in the rolled position.

Referring to FIGS. 18A-B, once the applicator 126 is expanded and/or unrolled, the hemostat 116 may be removed from the pocket 134 and placed against a target area, whereby one of the first or second portions 130, 132 of the applicator 126 may be used as a tamponade surface to tamponade down the hemostat 116. Using the applicator 126 may require a manual rotation of the applicator 126 about the axis of rotation (at the hinge pin 128), or a mechanism for the rotation (not shown) of the applicator 126 may be provided at the handle. As shown in FIG. 18B, for example, the applicator 126 has been rotated 90 degrees from its initial position after being expanded.

The applicator itself may include properties that are advantageous to the delivery, deployment, and tamponade of a hemostat 116. For example, in an alternative embodiment of an applicator 160 referring to FIG. 19, an aspect of one or both of the first and second portions 162, 164, of the applicator may include a cutout. As shown, the first portion 162 includes a cutout 165. The cutout 165 is advantageous in that it provides easier access via a grabbing instrument for a physician so that the clinician may grab and deploy the hemostat 116 to a target tissue. Notably, any of the embodiments as described herein may include one or more cutouts.

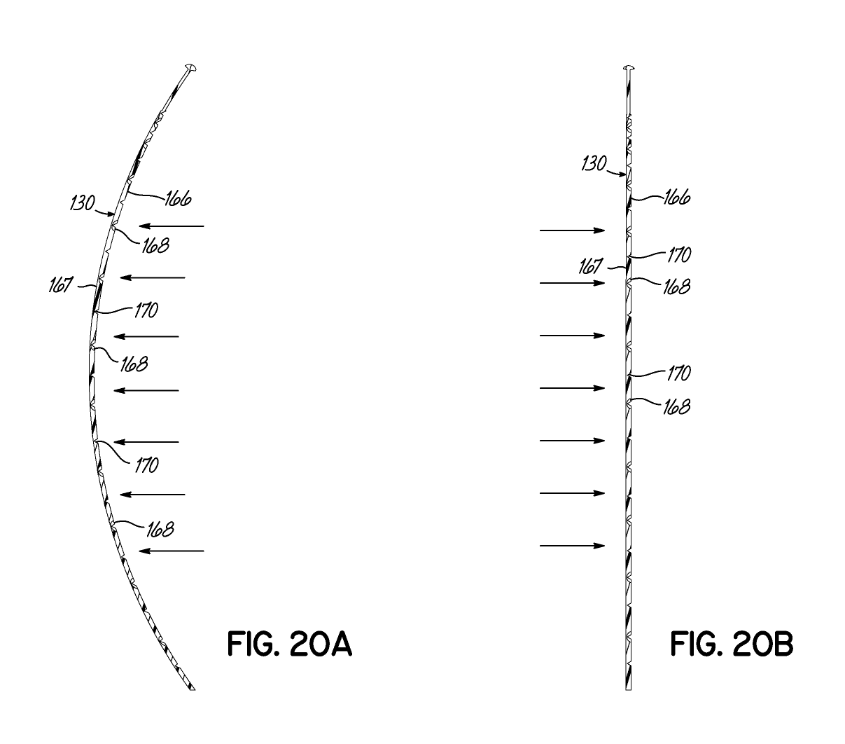

Referring back to the embodiment shown in FIGS. 12-15B, one or both of the first and second portions 130, 132 may include different properties. Referring specifically to FIGS. 20A-B, only the first portion 130 of the applicator 126 is shown and includes certain advantageous properties that provide for stiffness in one direction and flexibility in the other. More specifically, the first portion 130 of the applicator 126 includes a first side 166 and a second side 167. When the first side 166 is subjected to a first force in a first direction the first portion 130 of the applicator 126 is configured to bend a first amount. When the second side 167 is subjected to a second force that is substantially equal to the first force in a second direction that is substantially opposite to the first direction, the first portion 130 is configured to bend a second amount. The first amount is less than the second amount. As shown, these properties are provided by a series of cuts or dimples 168 along a series of ribs 170 (best seen in FIG. 14B). The dimples 168 allow ribs 170 to collapse in one direction, but provide stiffness in the opposite direction.

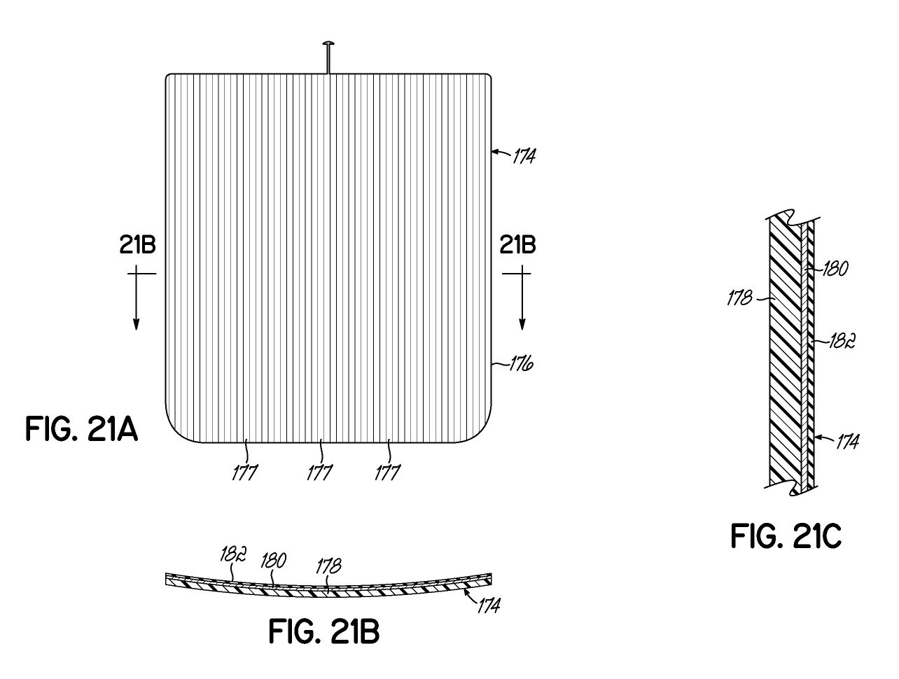

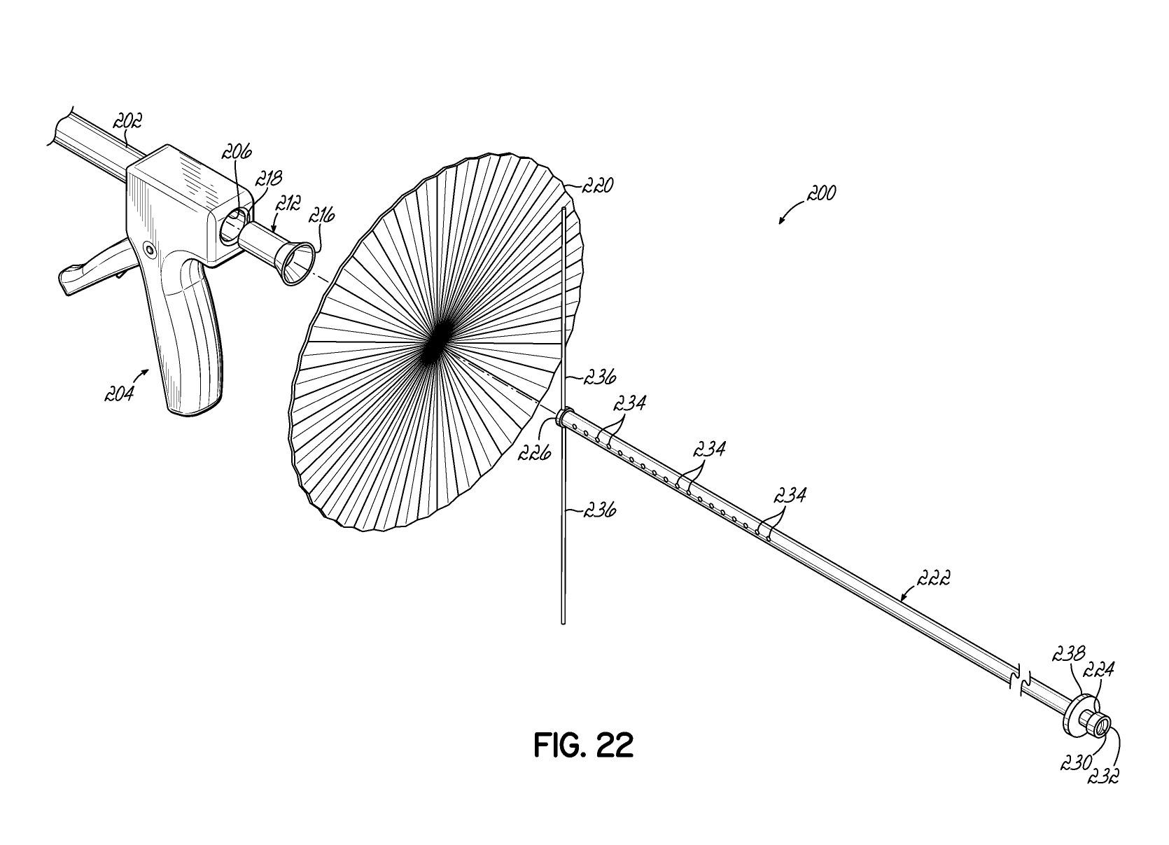

Referring to FIGS. 21A-C, another embodiment of an applicator 174 showing another ribbed configuration is shown (as also shown in FIGS. 17 and 19). Specifically, only the first portion 176 of the applicator 174 is shown. The first portion 176 may be used as a tamponade surface and may include three layers, some of which may be prestressed in order to give the applicator 174 its curved shape as shown. For example, the first layer 178 as shown may be cast polyurethane or similar material, the second layer 180 may be polyester thread that is prestressed with approximately 10 psi tension, and the third layer 182 may be polyester sheet that is also prestressed with approximately 10 psi tension. The ribbed embodiment shown (also shown in FIGS. 17 and 19) is advantageous in that the ribs 177 provide structural rigidity and are functional to reduce air or other bubbles between the hemostat 116 and target tissue. The ribbed configuration may be imparted to the flag due to the polyester fibers being embedded in the polyurethane. The applicator 174 in this embodiment may be flexible in one direction such that it may transition to the rolled-up or contracted state, and rigid in another direction such that it may be used as a tamponade surface to tamponade the hemostat 116 once deployed. In this and other embodiments, the second portion (not shown) may be MYLAR or other similar material.

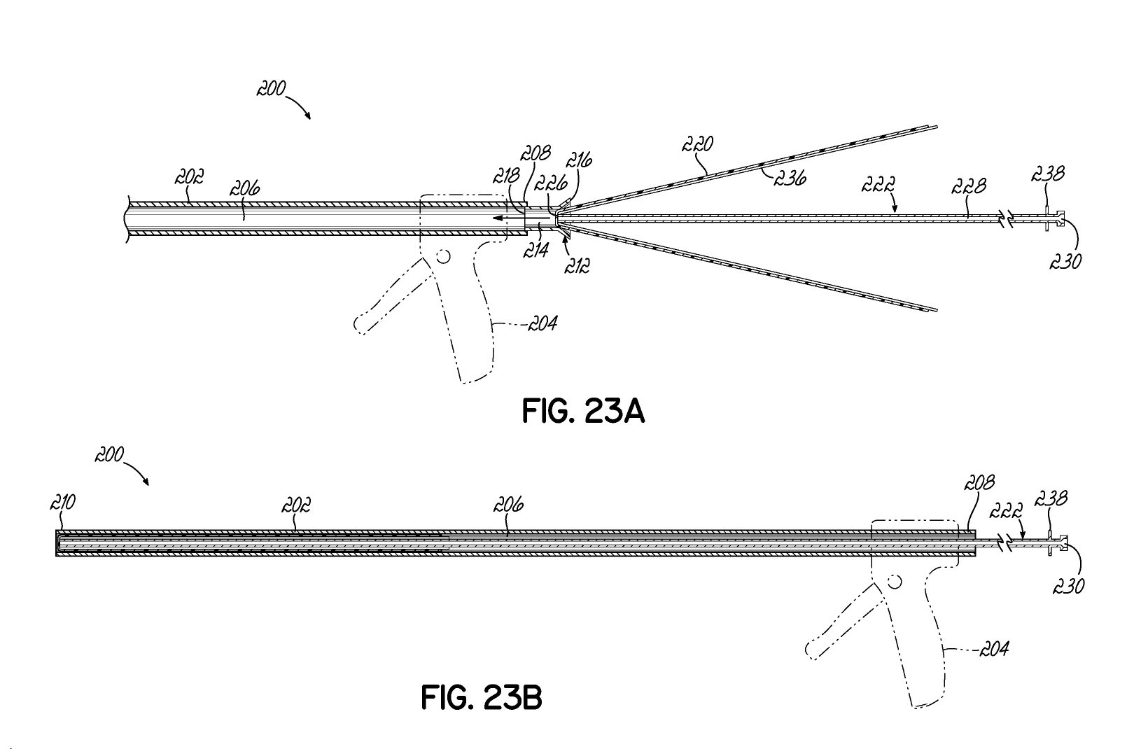

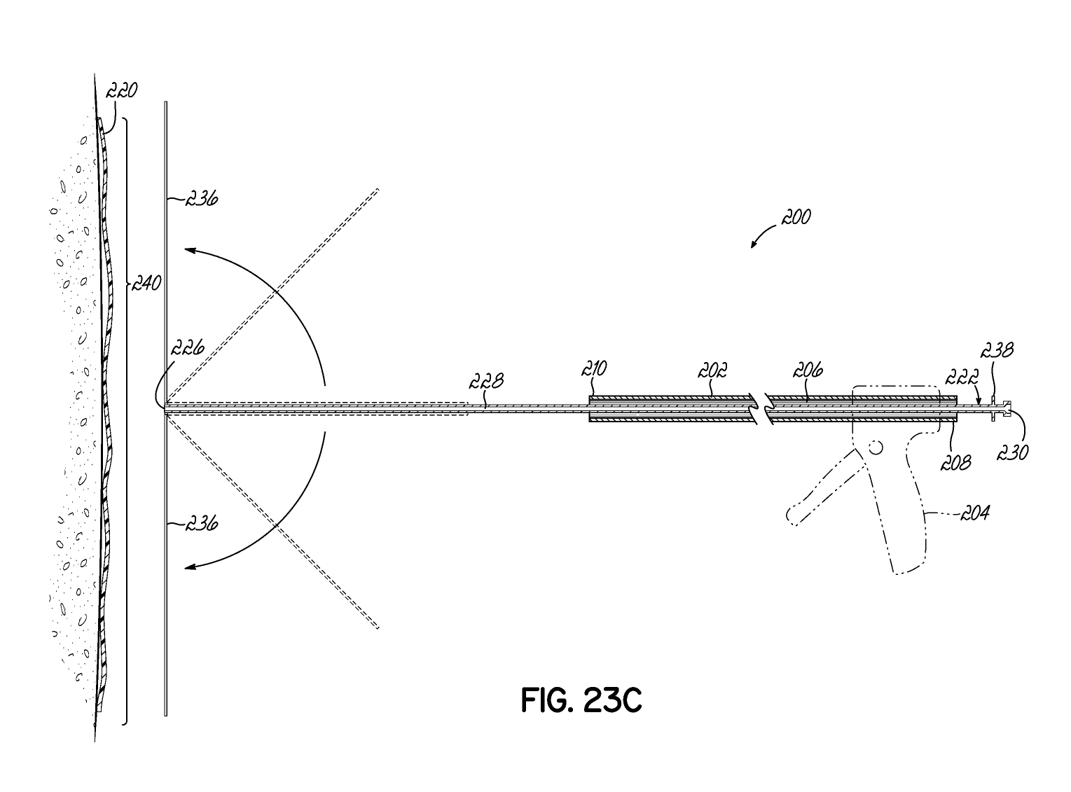

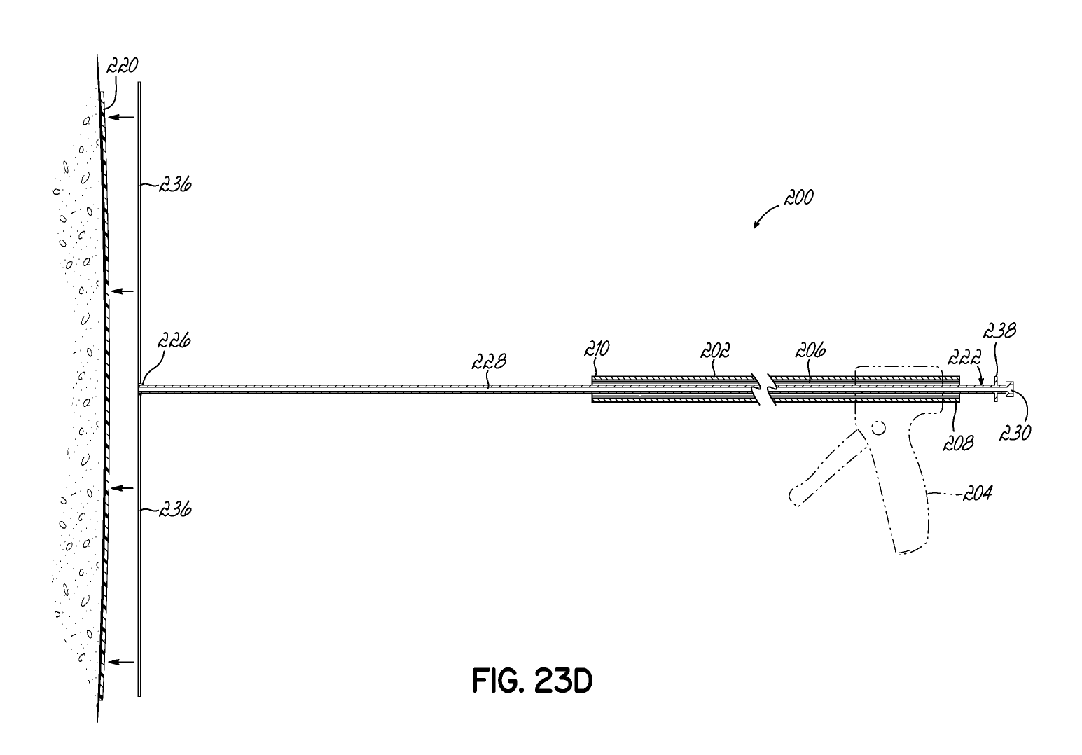

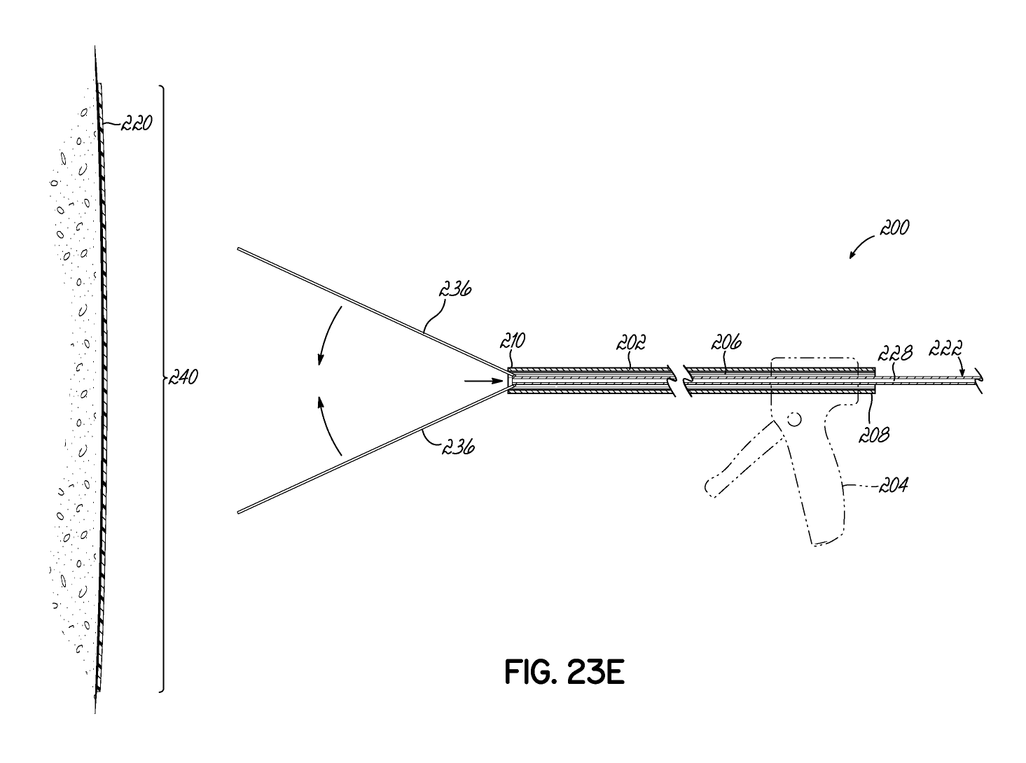

Referring to FIG. 22-23E, another embodiment of an instrument 200 is shown. The instrument includes a delivery cannula 202 having an optional handle and trigger mechanism 204 for selectively advancing other components of the instrument 200 within the lumen 206 of the delivery cannula 202. The lumen 206 extends between proximal and distal ends 208, 210 of the delivery cannula 202. In order to assist the collapsing and/or contracting of components into the lumen 206 of the delivery cannula 202, the instrument 200 includes an optional loading piece 212 having a lumen 214 extending between proximal and distal ends 216, 218. The distal portion of the loading piece 212 is of a generally cylindrical shape. The proximal portion is of a generally frustroconical shape. The distal portion is dimensioned to fit within the lumen 206 of the delivery cannula 202. As shown, the loading piece 212 is a separate piece, but it will be appreciated that the loading piece 212 may be integral with the delivery cannula 202. For example, the proximal end 208 of the delivery cannula 202 or handle and trigger mechanism 204 may include the frustroconical portion to allow the collapsing and/or contracting of components within the lumen 206.

The instrument 200 also includes a hemostat 220 (which may also not be considered part of the instrument 200 in some embodiments) and a stylet 222, which are loaded into the delivery cannula 202 in order to deliver the hemostat 220 to a target area of tissue, which will be described in more detail below. The hemostat 220 is shown as a generally circular shape, but it will be appreciated that the hemostat 220 may be other shapes or sizes, and that it may be cut to a desired shape or size by a clinician. The stylet 222, which may also be considered a shaft 114, includes a proximal end 224, a distal end 226, and a lumen 228 therebetween. The distal end 226 is shown to be closed. The proximal end 224 includes opening 230 with a luer fitting 232 so that the stylet 222 may be fluidicly communicated or connected with a source of fluid (not shown). The stylet 222 also includes a plurality of irrigation apertures 234 in order to impregnate the hemostat 220 with a liquid. At the distal end 226 of the stylet 222, a pair of collapsible arms 236 is provided in order to tamponade the hemostat 220 once deployed to a target tissue.

A flange 238 is provided near the proximal end 224 of the stylet 222 to provide an area for a clinician to grasp during use of the instrument 200, if needed. The flange 238 also acts as a stop to prevent the stylet 222 from traversing too far into the delivery cannula 202 and/or handle mechanism. In that regard, the cross-sectional dimension (i.e., diameter) of the flange 238 is greater than that of the lumen 206 of the delivery cannula 202.

In order to assemble the instrument 200 into the ready position, the hemostat 220 and stylet 222 are placed concentrically relative to loading device. The stylet 222 is pushed to force the hemostat 220 into a collapsed or contracted state. Similarly, and concurrently, the stylet 222, due to the flexibility of the arms 236, also is forced into a collapsed position. More specifically, as the hemostat 220 and stylet 222 are directed into the loading piece 212 and further into the lumen 206 of the delivery cannula 202, both the hemostat 220 and arms 236 of stylet 222 are folded in the proximal direction. The stylet 222 is then directed further into the lumen 206, thus also pushing the hemostat 220 towards the distal end 210 until the hemostat 220 and stylet 222 reach the ready position (FIG. 23B).

Preferably, the instrument 200 is assembled and in the ready position, including with the hemostat 220 and the stylet 222 collapsed within the delivery cannula 202, prior to being packaged and sent to a clinical environment for use by a clinician. Alternatively, the instrument 200 may arrive to the clinical environment in the disassembled state and a clinician may assemble the instrument 200 into the ready position. Notably, the instrument 200 may be reused after being used to deliver a hemostat 220, by simply inserting another hemostat 220 into the pocket and retracting the shaft within the delivery cannula 202, as described herein, such that the instrument 200 is again in the ready position.

In order to deliver and deploy the hemostat 220, the stylet 222 is moved axially in the distal direction relative to the delivery cannula 202. The stylet 222 may be moved in this manner by physically pushing the proximal end 224 of the stylet 222. Alternatively, and preferably, the stylet 222 is advanced by actuation of the trigger mechanism. Preferably, actuation of the trigger mechanism advances the stylet 222 in a metered manner, such that one “squeeze” of the trigger advances the stylet 222 a defined distance. The metered advancement is advantageous in that it allows the controlled deployment of the hemostat 220. In that regard, the clinician may advance the stylet 222 and hemostat 220 distally to a point where the ends of each arm are still within the lumen 206 of the delivery cannula 202. Advantageously, in one embodiment, the delivery cannula 202 may be transparent or translucent so that the clinician may visualize the stylet 222 and hemostat 220 within the cannula.

Once the clinician is ready to deploy the hemostat 220 to a target area, the clinician may essentially aim the distal end 210 of the delivery cannula 202 at the target area 240 and actuate the trigger mechanism 204 so that the arms 236 are no longer within the lumen 206. In a normal position, the arms 236 are in an unflexed position as shown in FIG. 22. Being collapsed within the lumen 206 in the proximal direction, as described herein, imparts a distal bias to the arms 236. Therefore, as shown in FIG. 23C, once the arms 236 leave the lumen 206, they are urged distally, back to the unflexed position. The hemostat 220 is thereby deployed to a target area 240 of tissue and likely may not be lying flat on the target area 240. Therefore, the arms 236 may be used as a tamponade to flatten the hemostat 220 against the target area 240. The arms 236 may be used to essentially scrape against the hemostat 220 in order to flatten the hemostat 220 and to eliminate any air or other fluid bubbles that may be between the hemostat 220 and the target area surface 240.

It may also be desirable in some situations to impregnate the hemostat 220 with a liquid, such as saline, growth hormones, or any other liquid discussed herein that may be desirable. To that end, stylet 222 is configured to receive a liquid and includes apertures 234, as disclosed above. In order to impregnate the hemostat 220 with liquid, the clinician fluidicly connects the stylet 222 to a source of fluid (not shown), such as a fluid-filled syringe (not shown). The fluid is then directed from the source into the lumen 228 of the stylet 222, whereby the fluid travels through the lumen 228 and out of the apertures 234, and onto and into the hemostat 220 that, in the collapsed or contracted position, is surrounding the portion of the stylet 222 that includes the apertures 234.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method and illustrative examples shown and described. Accordingly, departures may be from such details without departing from the scope or spirit of the general inventive concept.

What is claimed is:

1. An instrument for delivery, deployment, and tamponade of a hemostat, comprising:
a delivery cannula having a proximal end, a distal end, and a lumen therebetween;
a shaft having a distal end and being configured to be introduced into and move relative to the lumen; and
a hemostat applicator at the distal end of the shaft and including a tamponade surface, the applicator being configured to transition between a first, compact state for introduction into and movement within the lumen, and a second, expanded state as the applicator exits from the delivery cannula.

2. The instrument of claim 1, further comprising a plurality of resilient legs coupling the applicator and the distal end of the shaft, the legs configured to bias the applicator into the second, expanded state.

3. The instrument of claim 1, wherein rotating the shaft relative to the delivery cannula causes the applicator to exit from the delivery cannula.

4. The instrument of claim 1, wherein axial movement of the shaft relative to the delivery cannula causes the applicator to exit from the delivery cannula.

5. The instrument of claim 1, wherein the applicator is configured to articulate relative to the delivery cannula.

6. The instrument of claim 1, wherein the applicator further includes a pocket for receiving and storing the hemostat during delivery thereof.

7. The instrument of claim 1, wherein the shaft moves relative to the lumen along an axis distal end of the shaft is configured to articulate to a first angle relative to the axis.

8. An instrument for delivery, deployment, and tamponade of a hemostat, comprising:
a delivery cannula having a proximal end, a distal end, and a first lumen therebetween;
a shaft having a proximal end, a distal end, an outer wall between the proximal and distal ends, a second lumen between the proximal and distal ends, and an aperture traversing through the outer wall to the second lumen, the shaft being configured to be introduced into and move relative to the first lumen; and
a hemostat configured to transition between a first, compact state for introduction into and movement within the first lumen, and a second, expanded state and further configured to be carried by the shaft regardless of whether the hemostat is in the compact or expanded state;
wherein the hemostat is positioned relative to the aperture to receive fluid when the second lumen is in fluid communication with a source of fluid.

9. The instrument of claim 8, wherein the hemostat is positioned in a pocket of a hemostat applicator coupled to the distal end of the shaft.

10. The instrument of claim 9, wherein the applicator includes at least one fluid permeable portion to allow fluid to flow into the pocket.

11. The instrument of claim 8, further comprising a stylet having a proximal end, a distal end, an outer wall between the proximal and distal ends, a third lumen between the proximal and distal ends, and at least one aperture traversing through the outer wall to the third lumen, the stylet being configured to be introduced into and move relative to the second lumen.

12. A method for delivery, deployment, and tamponade of a hemostat using a delivery shaft having a hemostat applicator coupled to a distal end portion of the delivery shaft, the hemostat applicator including a pocket for receiving and carrying the hemostat, the method comprising:
inserting a delivery cannula having the delivery shaft therein into a trocar;
moving the delivery shaft relative to the delivery cannula until at least the applicator exits from the delivery cannula, whereupon the applicator transitions from a first, compact state to a second, expanded state; and
removing the hemostat from the pocket and deploying the hemostat to an area of tissue.

13. The method of claim 12, further comprising tamponading the hemostat and the area of tissue using the applicator.

14. The method of claim 12, wherein moving the delivery shaft relative to the delivery cannula includes rotating the delivery shaft relative to the delivery cannula.

15. The method of claim 12, wherein moving the delivery shaft relative to the delivery cannula includes moving the delivery shaft axially relative to the delivery cannula.

16. The method of claim 12, wherein the applicator biases from the compact state to the expanded state when the applicator begins to exit from a distal end of the delivery cannula.

17. The method of claim 12, further comprising fluidicly communicating the delivery shaft with a source of fluid.

18. The method of claim 17, further comprising impregnating the hemostat with the fluid.

19. The method of claim 17, further comprising applying the fluid to the area of tissue.

20. The method of claim 19, wherein the applying the fluid step is performed before deploying the hemostat to the area of tissue.

21. A method for delivery, deployment, and tamponade of a hemostat using a delivery shaft having a hemostat applicator coupled to a distal end portion of the delivery shaft, the hemostat applicator including a tamponade surface, the method comprising:
inserting a delivery cannula having the delivery shaft therein into a trocar;
moving the delivery shaft relative to the delivery cannula until at least the applicator exits from the delivery cannula, whereupon the applicator transitions from a first, compact state to a second, expanded state;
deploying a hemostat to an area of tissue; and
tamponading the hemostat and the area of tissue using the applicator

22. The method of claim 21, wherein moving the delivery shaft relative to the delivery cannula includes rotating the delivery shaft relative to the delivery cannula.

23. The method of claim 21, wherein moving the delivery shaft relative to the delivery cannula includes moving the delivery shaft axially relative to the delivery cannula.
